(19) United States
(12) Patent Application Publication
(10) Pub. No.: US 2021/0323918 A1
(43) Pub. Date: Oct. 21, 2021

(54) COMPOSITIONS AND METHODS FOR SUPPRESSING AND/OR TREATING A GROWTH RELATED DISEASE AND/OR A CLINICAL CONDITION THEREOF

(71) Applicant: CK Biotech Inc., Seoul (KR)

(72) Inventors: Kang-Yell Choi, Seoul (KR); Sehee Choi, Seoul (KR); Seol Hwa Seo, Seoul (KR); Eunhwa Kim, Seoul (KR)

(21) Appl. No.: 17/285,414

(22) PCT Filed: Oct. 14, 2019
(86) PCT No.: PCT/IB19/58749

§ 371 Date: Apr. 14, 2021

Related U.S. Application Data
(60) Provisional application No. 62/903,068, filed on Sep. 20, 2019.
(60) Provisional application No. 62/825,363, filed on Mar. 28, 2019.
(60) Provisional application No. 62/799,921, filed on Feb. 1, 2019.
(60) Provisional application No. 62/799,912, filed on Feb. 1, 2019.

(30) Foreign Application Priority Data
Oct. 15, 2018 (KR) 10-2018-0122511

(51) Int. Cl. C07D 209/34; A61P 19/08; C07D 513/14; C07D 221/18; C07C 237/48; C07D 487/22; G01N 33/68; A61K 9/00

(52) U.S. Cl. CPC C07D 209/34; A61P 19/08; C07D 513/14; C07D 221/18; C07C 237/48; C07D 487/22; G01N 2800/7014; A61K 9/0053; A61K 9/0019; C07C 2603/44; G01N 2800/10; G01N 2800/52; G01N 33/6893

(57) ABSTRACT

Therapeutic compositions comprising one or more agents that inhibit CXXC5-DVL interface, and methods of administering those therapeutic compositions to model, treat, reduce resistance to treatment, prevent, and diagnose a condition/disease associated with growth or a related clinical condition thereof, are disclosed.

### CROSS-REFERENCE TO RELATED APPLICATIONS

This application claims the benefit of Korean Application No. KR 10-2018-0122511, filed Oct. 15, 2018, the U.S. provisional application No. 62/799,921, filed Feb. 1, 2019, the U.S. provisional application No. 62/799,912, filed Feb. 1, 2019, the U.S. provisional application No. 62/825,363, filed Mar. 28, 2019, and the U.S. provisional application No. 62/903,068, filed Sep. 20, 2019, the entire disclosures of all of which are hereby expressly incorporated by reference herein.

### FIELD

Various aspects and embodiments disclosed herein relate generally to the modelling, treating, reducing resistance to a treatment, preventing, and diagnosing of a condition/disease associated with growth or a related clinical condition thereof. Embodiments include compositions and methods for treating the condition/disease, comprising providing to a subject at least one therapeutically effective dose of a compound and/or composition disclosed herein. Other embodiments include methods for altering and/or suppressing the activity of the CXXC5-DVL interface in a subject.

### BACKGROUND

Longitudinal bone growth takes place in growth plates, which are comprised of a thin layer of transient cartilage tissue. Chondrocytes in this cartilage layer proliferate and undergo hypertrophic differentiation followed by apoptosis and subsequent remodeling into bone tissue, resulting in bone elongation. Longitudinal bone growth occurs rapidly during fetal development and early childhood; bone growth gradually slows and eventually ceases at the end of puberty in part due to growth plate senescence. Currently, many children undergo early pubertal development, which leads to an earlier growth plate senescence. These phenomena, known as precocious puberty, reveals premature termination of longitudinal bone growth, resulting in short adult stature. The exact mechanisms involving regulation of growth plate senescence are currently unknown.

Some studies have reported the involvement of Wnt/β-catenin signaling in growth plate maturation. CXXC finger protein 5 (CXXC5) is a negative regulator of Wnt/β-catenin signaling, functioning via interaction with the PDZ domain of dishevelled (DVL) in the cytosol. Inhibition of the CXXC5-DVL interaction improved several pathophysiological phenotypes involving Wnt/β-catenin signaling including osteoporosis, cutaneous wounds, and hair loss through activation of the Wnt/β-catenin. Due to the complexity of the processes involving regulation of growth plate senescence and/or related conditions thereof, development of a new treatment regimen that would enhance longitudinal bone growth in a subject is much needed.

### SUMMARY

Given CXXC5's role as a negative regulator of Wnt/β-catenin signaling, it is an attractive target for the development of compounds that can interfere with its activity. Some aspects of the instant disclosure include compounds that interfere with CXXC5-DVL interface and methods of using the same to influence the growth of a subject.

Some embodiments of the instant application relate to compositions and methods for treating a condition and/or disease associated with growth or a related clinical condition in a subject. In certain embodiments, the compositions and methods disclosed herein involve suppression of one or more side effects of a therapeutic regime. Other embodiments relate to compositions and methods for treating a subject diagnosed with a disease or having a condition contributed to early pubertal development, caused at least in part by earlier growth plate senescence.

In a first aspect, compositions disclosed herein comprise at least one agent that inhibits the CXXC5-DVL interface—the interface between CXXC finger protein 5 (CXXC5) and dishevelled (DVL)—in a subject. In some embodiments, at least one agent that inhibits CXXC5-DVL interface comprises at least one agent that binds to the PDZ domain of dishevelled (DVL) and/or the DVL binding motif, and/or at least one GSK3β inhibitor, or a combination thereof.

A first embodiment includes a compound of Formula I,

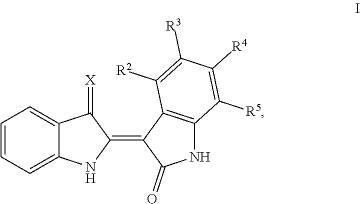

wherein X is O or N optionally substituted with R¹;

R¹ is hydrogen, hydroxy, alkyl, alkenyl, or an alkoxy optionally substituted with alkyl, alkenyl, haloalkyl, aryl, or benzyl; or R¹ is hydrogen, alkyl, alkenyl, or an alkoxy substituted with butyl, alkenyl, haloalkyl, aryl, or benzyl;

R², R³, R⁴ and R⁵ are independently hydrogen, nitro, halogen, alkyl, alkenyl, haloalkyl, alkoxy, haloalkoxy, or a carboxy.

A second embodiment includes the compound according to the compound of the first embodiment, wherein X is O.

A third embodiment includes the compound according to the compound according to the compound of the first embodiment, wherein X is N and R¹ is hydroxy or alkoxy optionally substituted with alkyl, alkenyl, haloalkyl, aryl, or benzyl.

A fourth embodiment includes the compound according to the compound according to any one of the first to the third embodiments, wherein R¹ is alkoxy optionally substituted with alkyl, alkenyl, haloalkyl, aryl, or benzyl.

A fifth embodiment includes the compound according to the compound according to any one of the first to the fourth embodiments, wherein the compound is any one of the compounds disclosed in FIG. 14, FIG. 15, FIG. 16, Table 6, Table 7, and/or Table 8.

A sixth embodiment includes the compound according to any one of the first to the fifth embodiments, wherein the compound is at least one compound comprising

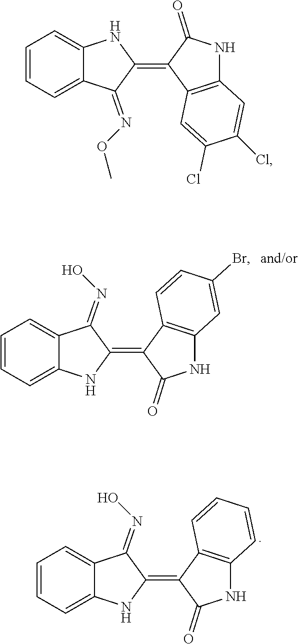 [SMILES: CO/N=C1/C(=C2/C(=O)Nc3cc(Cl)c(Cl)cc32)Nc2ccccc21 ; O=C1Nc2cc(Br)ccc2/C1=C1/Nc2ccccc2/C1=N\O ; O=C1Nc2ccccc2/C1=C1/Nc2ccccc2/C1=N\O]

A seventh embodiment includes a compound of Formula II,

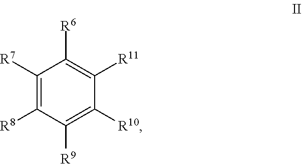

wherein R⁶, R⁷, R⁸, R⁹, and R¹⁰ are independently hydrogen, halogen, hydroxy, alkyl, haloalkyl, alkoxy, or

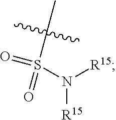

R¹¹ is C₁-C₆ alkyl, C₁-C₆ alkenyl, N, diimide, each substituted with R¹²,

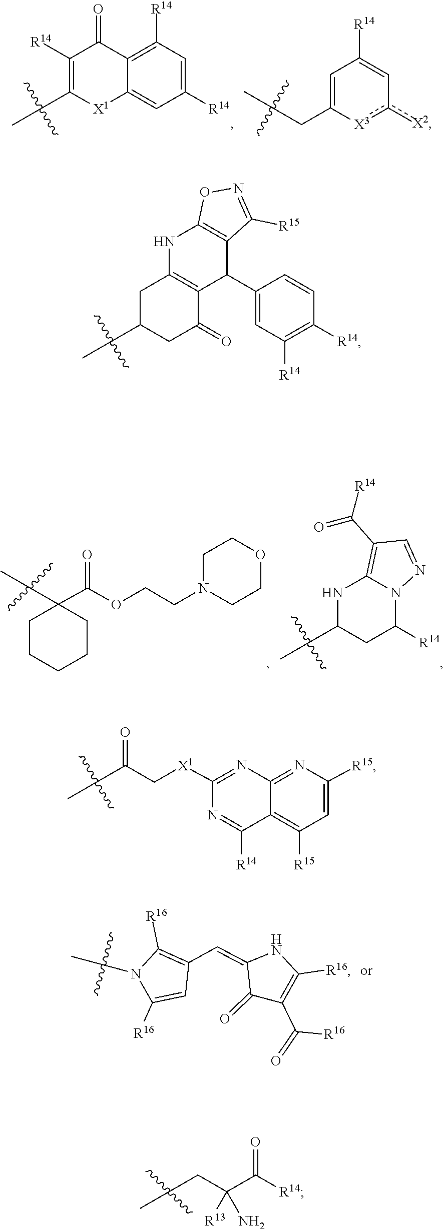

or

R¹¹ is

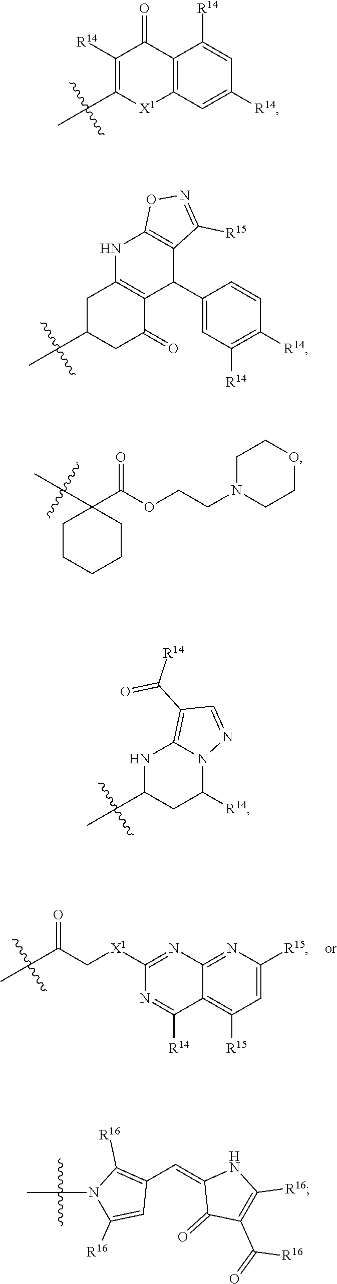

R¹² is

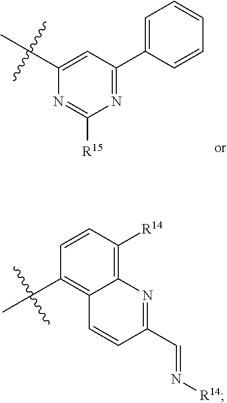

R¹³ is hydrogen or an alkyl optionally substituted with hydrogen, halogen, hydroxy, alkoxy, or haloalkyl;

each R¹⁴, each R¹⁵, and each R¹⁶ are independently hydrogen; halogen; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl;

X¹, X² and X³ are independently carbon, nitrogen, oxygen, or sulfur.

An eighth embodiment includes the compound according to the seventh embodiment, wherein R¹¹ is N or diimide, each substituted with R¹².

A ninth embodiment includes the compound according to any one of the seventh to the eighth embodiments, wherein R¹¹ is

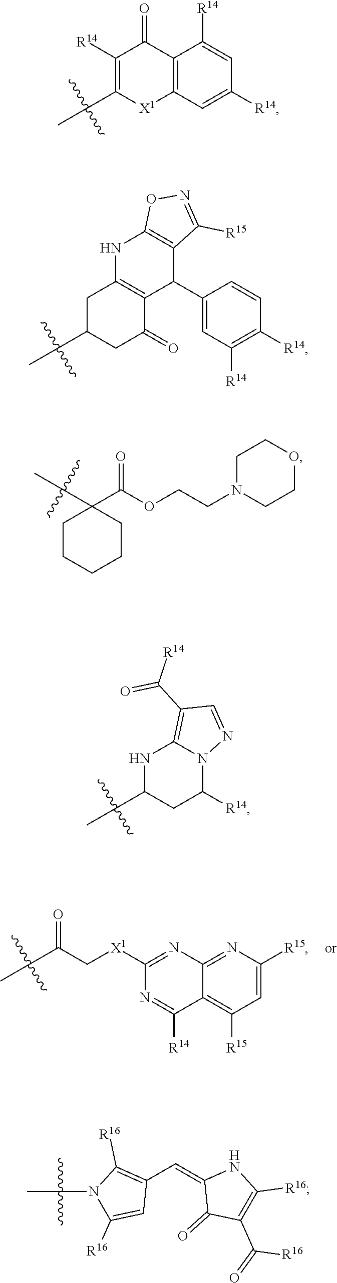

A tenth embodiment includes the compound according to any one of the seventh to the ninth embodiments, wherein the compound is

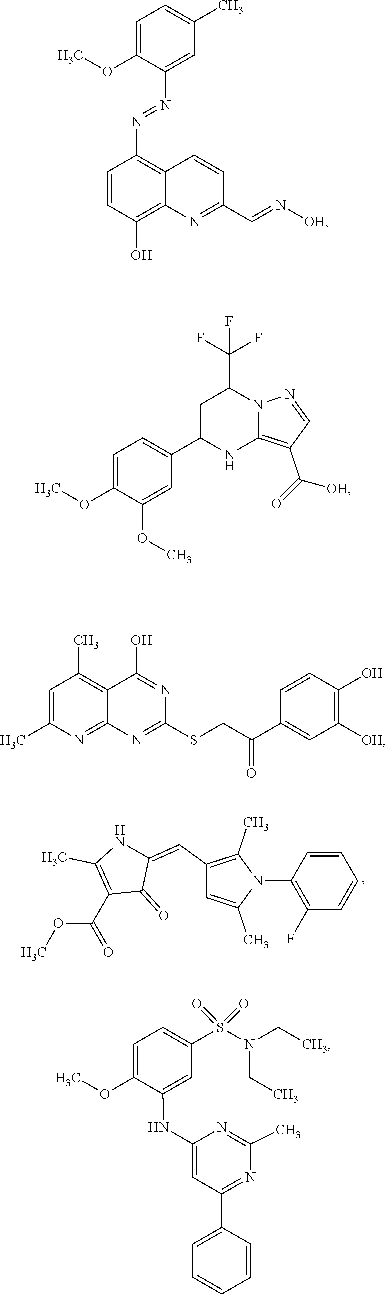 [SMILES: COc1ccc(C)cc1/N=N/c1ccc(O)c2nc(/C=N/O)ccc12 ; COc1ccc(C2CC(C(F)(F)F)n3ncc(C(=O)O)c3N2)cc1OC ; Cc1cc(C)c2c(O)nc(SCC(=O)c3ccc(O)c(O)c3)nc2n1 ; COC(=O)C1=C(C)N/C(=C/c2cc(C)n(-c3ccccc3F)c2C)C1=O ; CCN(CC)S(=O)(=O)c1ccc(OC)c(Nc2cc(-c3ccccc3)nc(C)n2)c1]

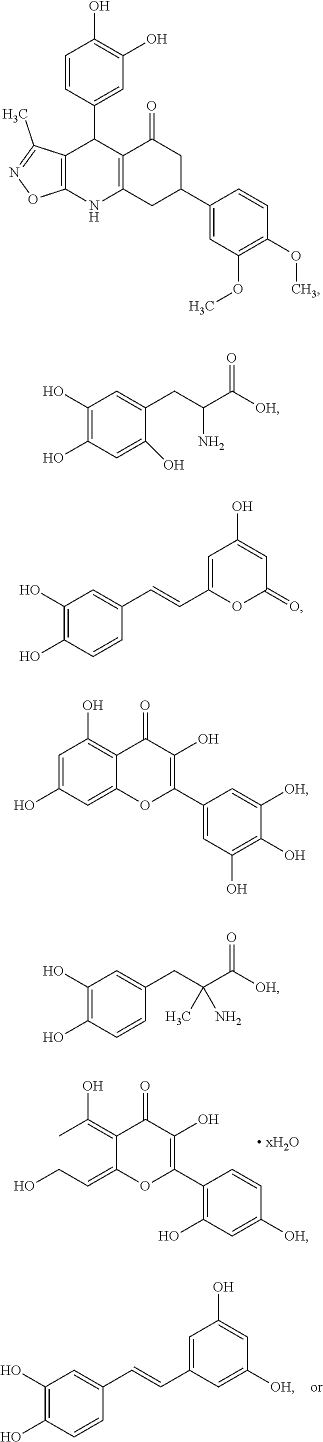

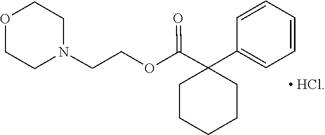 [SMILES: O=C(OCCN1CCOCC1)C1(c2ccccc2)CCCCC1]

An eleventh embodiment includes a compound of Formula III,

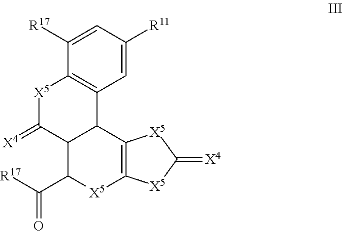

wherein each R¹⁷ is independently hydrogen; halogen; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl;

X⁴ and X⁵ are independently nitrogen, oxygen, or sulfur.

A twelfth embodiment includes the compound according to the eleventh embodiment, wherein each R¹⁷ is independently halogen or hydroxy.

A thirteenth embodiment includes the compound according to any one of the eleventh to the twelfth embodiments, wherein the compound is

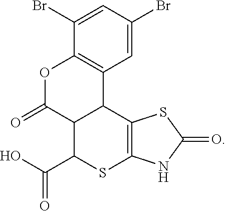 [SMILES: O=C(O)C1Sc2[nH]c(=O)sc2C2c3cc(Br)cc(Br)c3OC(=O)C12]

A fourteenth embodiment includes a compound of Formula IV,

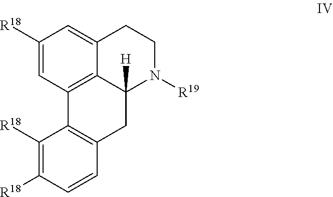

wherein each R¹⁸ and R¹⁹ are independently hydrogen; hydroxy; halogen; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl.

A fifteenth embodiment includes the compound according to the fourteenth embodiments, wherein R¹⁹ is alkyl optionally substituted with hydrogen, halogen, hydroxy, alkoxy, or haloalkyl.

A sixteenth embodiment includes the compound according to any one of fourteenth to the fifteenth embodiments, wherein each R¹⁸ is independently hydrogen, hydroxy, halogen, alkoxy, alkyl, alkenyl, or haloalkyl.

A seventeenth embodiment includes the compound according to any one of fourteenth to the sixteenth embodiment, wherein the compound is

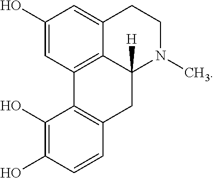

An eighteenth embodiment includes a compound of Formula V,

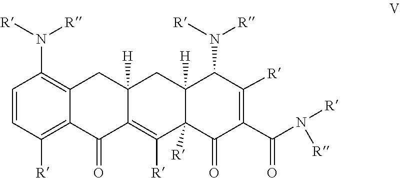

wherein each R′ and each R″ are independently hydrogen; halogen; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl.

A nineteenth embodiment includes the compound according to the eighteenth embodiment, wherein each R′ and each R″ are independently hydrogen, halogen, hydroxy, alkyl, alkenyl, alkoxy, or haloalkyl.

A twentieth embodiment includes the compound according to any one of the eighteenth to the nineteenth embodiments, wherein the compound is

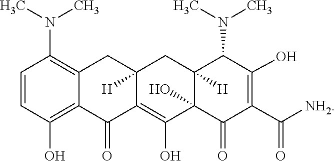

A twenty first embodiment includes a compound of Formula VI,

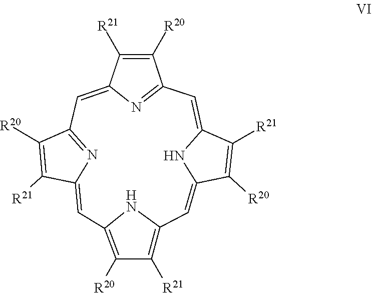

wherein each R²⁰ and each R²¹ are independently hydrogen; halogen; haloalkyl optionally substituted with hydrogen, halogen, hydroxy, or alkoxy; alkyl optionally substituted with hydrogen, halogen, hydroxy, alkoxy, haloalkyl, or a carbonyl, optionally substituted with hydrogen, halogen, alkyl, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl.

A twenty second embodiment includes the compound according to the twenty first embodiment, wherein each R²⁰ and each R²¹ are independently hydrogen, halogen, hydroxy, alkyl, alkenyl, alkoxy, carbonyl, carboxyl, or haloalkyl.

A twenty third embodiment includes the compound according to any one of the twenty first to the twenty second embodiments, wherein the compound is

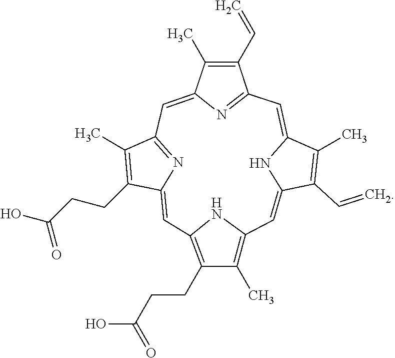 [SMILES: C=CC1=C(C)c2cc3nc(cc4[nH]c(cc5[nH]c(cc1n2)c(C)c5C=C)c(C)c4CCC(=O)O)C(CCC(=O)O)=C3C]

A twenty fourth embodiment includes at least one of the compounds according to any one of the first to the twenty third embodiments, wherein the compound inhibits or reduces the CXXC5-DVL interface, the interaction between CXXC5 and DVL, and/or the activity of CXXC5 and/or the CXXC5-DVL interface.

A twenty fifth embodiment includes at least one of the compounds according to any one of the preceding embodiments, wherein the compound inhibits or reduces the interaction between CXXC5 and DVL by directly competing with CXXC5 for a binding site in DVL, by directly binding to DVL, and/or by directly binding to the PZD domain of DVL.

A twenty sixth embodiment includes a pharmaceutical composition comprising at least one compound according to any one of the first to the twenty fifth embodiments and/or a pharmaceutically acceptable hydrate, salt, metabolite, or carrier thereof.

In a second aspect, methods disclosed herein include methods of treating at least one clinical condition, comprising administering to a subject at least one therapeutically effective dose of any of the compositions disclosed herein. The subject can be diagnosed with a clinical condition selected from and/or comprising a growth-related disease or a similar condition thereof In certain embodiments, the methods disclosed herein further comprise administering to the subject at plurality of therapeutically effective doses of any of the compositions disclosed herein.

A twenty seventh embodiment includes a method of treating a growth-related disease or a similar condition, comprising: administering to a subject at least one therapeutically effective dose of at least one agent that inhibits or reduces the CXXC5-DVL interface the interaction between CXXC5 and DVL, and/or the activity of the CXXC5 and/or the CXXC5-DVL interface; and/or administering to a subject at least one therapeutically effective dose of at least one agent comprising at least one compound according to any one of the first to the twenty fifth embodiments and/or at least one composition according to the twenty sixth embodiment.

A twenty eighth embodiment includes the method according to the twenty seventh embodiment, further comprising the step of: detecting an upregulated expression of CXXC5 in the subject.

A twenty ninth embodiment includes the method according to any one of the twenty seventh to the twenty eighth embodiments, further comprising the step of: identifying the subject at risk for a growth-related disease or a similar condition.

A thirtieth embodiment includes at least one of the methods according to any one of the twenty seventh to the twenty ninth embodiments, wherein the growth-related disease or a similar condition includes at least one condition selected from, or comprising, growth disorders, human growth hormone deficiency, Cushing's Syndrome, hypothyroidism, nutritional short stature, intrauterine growth retardation, Russell Silver syndrome, disproportionate short stature, achondroplasia, growth related disorders, poor nutrition and systemic diseases, bone disorders, and/or precocious puberty.

A thirty first embodiment includes at least one of the methods according to any one of the twenty seventh to the thirtieth embodiments, wherein the subject exhibits abnormal growth plate senescence. Consistent with these embodiments, the abnormal growth plate senescence includes earlier than normal growth plate senescence in a subject and/or a treatment-induced growth plate senescence in a subject.

A thirty second embodiment includes at least one of the methods according to any one of the twenty seventh to the thirty first embodiments, wherein the subject is diagnosed with a growth disorder and/or precocious puberty.

A thirty third embodiment includes at least one of the methods according to any one of the twenty seventh to the thirty second embodiments, wherein the at least one agent that inhibits the CXXC5-DVL interface, that inhibits the interaction between CXXC5 and DVL, and/or that inhibits the activity of CXXC5 and/or the CXXC5-DVL interface comprises at least one compound according to any one of the first to the twenty fifth embodiments and/or at least one composition according to the twenty sixth embodiment.

A thirty fourth embodiment includes at least one of the methods according to the thirty third embodiments, wherein the method further includes the step of: administering at least one therapeutically effective dose of at least one additional agent comprising a GSK3β inhibitor and/or an inhibitor of Wnt/β-catenin pathway.

A thirty fifth embodiment includes at least one of the methods according to any one of the twenty seventh to the thirty fourth embodiments, wherein the subject is a human adult, a human child, and/or an animal.

A thirty sixth embodiment includes at least one of the methods according to any one of the twenty seventh to the thirty fifth embodiments, wherein the at least one agent and/or the at least one additional agent is administered orally or intravenously.

A thirty seventh embodiment includes at least one of the methods according to any one of the twenty seventh to the thirty sixth embodiments, wherein the therapeutically effective dose of at least one compound according to any one of the first to the twenty fifth embodiments and/or at least one composition according to the twenty sixth embodiment, is on the order of between about 5 mg to about 2000 mg and the dose of the compound is administered to the subject at least once per day. In some embodiments, the therapeutically effective dose of at least one compound according to any one of the first to the twenty fifth embodiments and/or at least one composition according to the twenty sixth embodiment, includes, but is not limited to, on the order of between: about 10 mg to about 1900 mg; about 15 mg to about 1800 mg; about 15 mg to about 1700 mg; about 20 mg to about 1600 mg; about 25 mg to about 1500 mg; about 30 mg to about 1000 mg; about 50 mg to about 1000 mg; about 50 mg to about 800 mg; about 100 mg to about 800 mg; about 300 mg to about 800 mg; about 500 mg to about 800 mg; about 5 mg to about 50 mg; about 1000 mg to about 1700 mg; about 1200 mg to about 1700 mg; about 1500 mg to about 1700 mg; about 10 mg to about 1000 mg; about 10 mg to about 30 mg; about 1500 mg to about 2000 mg; about 100 mg to about 200 mg; about 100 mg to about 150 mg; and/or any combination thereof. Consistent with these embodiments, the therapeutically effective dose of at least one compound according to any one of the first to the twenty fifth embodiments and/or at least one composition according to the twenty sixth embodiment, includes, but not limited to, on the order of between: about 1 mg/m2 to about 1500 mg/m2; about 10 mg/m2 to about 1000 mg/m2; about 20 mg/m2 to about 800 mg/m2; about 10 mg/m2 to about 50 mg/m2; about 800 mg/m2 to about 1200 mg/m2; about 50 mg/m2 to about 500 mg/m2; about 500 mg/m2 to about 1000 mg/m2; about 80 mg/m2 to about 150 mg/m2; about 80 mg/m2 to about 120 mg/m2; and/or any combination thereof.

A thirty eighth embodiment includes at least one of the methods according to any one of the twenty seventh to the thirty sixth embodiments, wherein the therapeutically effective dose of at least one compound according to any one of the first to the twenty fifth embodiments and/or at least one composition according to the twenty sixth embodiment, is on the order of between about 0.01 mg to about 200 mg and the dose of the compound is administered to the subject at least once per day. In some embodiments, the therapeutically effective dose of at least one compound according to any one of the first to the twenty fifth embodiments and/or at least one composition according to the twenty sixth embodiment, includes, but is not limited to, on the order of between: about 0.01 mg to about 150 mg; about 0.01 mg to about 100 mg; about 0.01 mg to about 80 mg; about 0.01 mg to about 60 mg; about 0.05 mg to about 100 mg; about 0.05 mg to about 80 mg; about 0.05 mg to about 50 mg; about 0.1 mg to about 100 mg; about 0.1 mg to about 50 mg; about 0.2 mg to about 100 mg; about 0.2 mg to about 50 mg; about 0.5 mg to about 100 mg; about 0.5 mg to about 50 mg; about 100 mg to about 200 mg; ; about 100 mg to about 150 mg; and/or any combination thereof. In some of these embodiments, the therapeutically effective dose of at least one compound according to any one of the first to the twenty fifth embodiments and/or at least one composition according to the twenty sixth embodiment, includes, but not limited to, on the order of between: about 0.01 mg/m²to about 100 mg/m²; about 0.01 mg/m² to about 80 mg/m²; about 0.01 mg/m² to about 50 mg/m²; about 0.01 mg/m² to about 25 mg/m²; about 0.05 mg/m² to about 100 mg/m²; about 0.05 mg/m² to about 80 mg/m²; about 0.05 mg/m² to about 50 mg/m²; about 80 mg/m² to about 150 mg/m²; about 80 mg/m² to about 120 mg/m²; and/or any combination thereof

In a third aspect, methods provided by the present application reduce and/or suppress a side effect of a therapeutic regime, the methods comprising administering to a subject at least one therapeutically effective dose of at least one agent that inhibits or reduces the CXXC5-DVL interface in a subject; and/or administering to a subject at least one therapeutically effective dose of at least one agent comprising at least one compound according to any one of the first to the twenty fifth embodiments and/or at least one composition according to the twenty sixth embodiment; wherein the subject has received at least one therapeutic regime selected from growth hormone and/or sex hormone therapy, surgical treatment, and/or combinations thereof, and wherein the subject experiences at least one side effect as a consequence of the therapeutic regime. Consistent with these embodiments, side effects can include, but are not limited to, drug-resistance, relapse, inflammation, or any combination thereof.

A thirty ninth embodiment includes a method of detecting one or more growth-related disease markers, comprising: providing a sample of blood, cells, or tissue from a subject suspected of having a growth-related disease or condition; and detecting upregulation in one or more markers in the sample, wherein the one or more markers comprise estrogen and/or CXXC5.

A fortieth embodiment includes the method according to the thirty ninth embodiment, wherein the growth-related disease includes at least one condition selected from, or comprising, growth disorders, human growth hormone deficiency, Cushing's Syndrome, hypothyroidism, nutritional short stature, intrauterine growth retardation, Russell Silver syndrome, disproportionate short stature, achondroplasia, growth related disorders, poor nutrition and systemic diseases, bone disorders, and/or precocious puberty. Consistent with these embodiments, CXXC5 is overexpressed in the growth plate of the subject at least about 10%, about 20%, about 25%, about 30%, about 40%, about 50%, about 60%, about 70%, about 80%, about 90%, about 100%, about 200%, about 300%, about 400%, about 500%, and/or about 1000%, or any combination thereof, as compared to that of a normal subject known not to have a growth related disease; and/or CXXC5 is overexpressed in the growth plate of the subject at least about 1.5 fold, about 2 fold, about 2.5 fold, about 3 fold, about 3.5 fold, about 4 fold, about 4.5 fold, about 5 fold, about 6 fold, about 7 fold, about 8 fold, about 9 fold, about 10 fold, about 15 fold, about 20 fold, about 25 fold, about 50 fold, and/or about 100 fold, or any combination thereof, as compared to that of a normal subject known not to have a growth related disease.

A forty first embodiment includes at least one of the methods according to the thirty ninth to the fortieth embodiments, further including the step of: treating the subject using at least one method according to any one of the twenty seventh to the thirty eighth embodiments.

A forty second embodiment includes a method of suppressing the activity of CXXC5, comprising the steps of: providing a subject at least one therapeutically effective dose of at least one compound according to any the first to the twenty fifth embodiments, or a pharmaceutically acceptable salt thereof, or a metabolite thereof.

A forty third embodiment includes the method according to the forty second embodiment, wherein the subject comprises a human, an animal, a cell, and/or a tissue. Consistent with these embodiments, the cell includes at least one type cells including chondrocytes, osteoblasts, osteoclasts, osteocytes, and osteoprogenitor (or osteogenic) cells.

A forty fourth embodiment includes a kit for for carrying out any one of the preceding methods disclosed herein. Components of the kit include, but are not limited to, one or more of agents/compositions disclosed herein, reagents, containers, equipment and/or instructions for using the kit.

A forty fifth embodiment includes the kit according to the forty fourth embodiment, wherein the one or more of agents/compositions includes at least one compound according to any one of the first to the twenty fifth embodiments and/or at least one composition according to the twenty sixth embodiment.

A forty sixth embodiment includes a method of determining the presence of a growth related disease in a subject, the method comprising assaying for a level of expression of CXXC5 gene and/or a level of expression of CXXC5 protein that is elevated as compared to a reference value.

A forty seventh embodiment includes the method according to the forty sixth embodiment, wherein the growth related disease includes at least one condition selected from, or comprising, includes at least one condition selected from, or comprising, growth disorders, human growth hormone deficiency, Cushing's Syndrome, hypothyroidism, nutritional short stature, intrauterine growth retardation, Russell Silver syndrome, disproportionate short stature, achondroplasia, growth related disorders, poor nutrition and systemic diseases, bone disorders, and/or precocious puberty; wherein the growth related disease includes at least one condition selected from, or comprising, growth related disorders, poor nutrition and systemic diseases, bone disorders, and/or precocious puberty; and/or wherein the growth related disease includes precocious puberty.

A forty eighth embodiment includes the method according to any one of the forty sixth to the forty seventh embodiments, wherein the reference value is the level of expression of CXXC5 gene or the level of expression of CXXC5 protein in a normal subject known not to have a growth related disease.

A forty ninth embodiment includes the method according to any one of the forty sixth to the forty eighth embodiments, wherein the level of expression of CXXC5 gene and/or the level of expression of CXXC5 protein that is elevated in the subject, growth plate of the subject, and/or chondrocytes in the growth plate of the subject.

A fiftieth embodiment includes the method according to any one of the forty sixth to the forty ninth embodiments, wherein the level of expression of CXXC5 gene and/or the level of expression of CXXC5 protein is elevated at least about 10%, about 20%, about 25%, about 30%, about 40%, about 50%, about 60%, about 70%, about 80%, about 90%, about 100%, about 200%, about 300%, about 400%, about 500%, and/or about 1000%, or any combination thereof, as compared to the reference value; and/or wherein the level of expression of CXXC5 gene and/or the level of expression of CXXC5 protein is elevated at least about 1.5 fold, about 2 fold, about 2.5 fold, about 3 fold, about 3.5 fold, about 4 fold, about 4.5 fold, about 5 fold, about 6 fold, about 7 fold, about 8 fold, about 9 fold, about 10 fold, about 15 fold, about 20 fold, about 25 fold, about 50 fold, and/or about 100 fold, or any combination thereof, as compared to the reference value.

A fifty first embodiment includes the method according to any one of the forty sixth to the fiftieth embodiments, further comprising: treating the subject using at least one method according to any one of the twenty seventh to the thirty eighth embodiments.

### BRIEF DESCRIPTION OF THE DRAWINGS

The following drawings form part of the present specification and are included to further demonstrate certain embodiments. Some embodiments may be better understood by reference to one or more of these drawings alone or in combination with the detailed description of specific embodiments presented.

FIG. 1A. Graph illustrating gene set enrichment analysis (GSEA) of microarray transcriptome data from the proliferative zone of growth plates in 3- and 12-week-old rats (GEO: GSE16981) for Wnt/β-catenin signaling-activated gene signatures (upper, MSigDB: M11722 and lower, MSigDB: M2680) (n=5). NES, normalized enrichment score; ES, enrichment score; FDR, false discovery rate.

FIG. 1B. Graph illustrating the relative expression changes of Cxxc5 in 3-, 6-, 9-, and 12-week-old rat growth plates (GEO: GSE16981) (mean±s.e.m., n=5, *P<0.05 and **P<0.005 versus 3-week-old).

FIG. 1C. Graph illustrating qRT-PCR analyses of relative mRNA expression of Cxxc5 and Runx2 in the growth plate of proximal tibiae of 3-, 6-, 9-, and 12-week-old mice (mean±s.e.m., n=3, ###P<0.0005 and *P<0.05 versus 3-week-old).

FIG. 1D. Immunoblot analyses illustrating the expression of the indicated proteins in the growth plate of proximal tibiae of 3-, 6-, 9-, and 12-week-old mice.

FIG. 1E. Immunohistochemical analyses illustrating the expression of the indicated proteins in the growth plate of proximal tibiae of 3-, 6-, 9-, and 12-week-old mice (left) and quantitative analyses (mean±s.e.m., n=3, * or #P<0.05, and **P<0.005 versus 3-week-old) (right). Scale bars, 50 μm.

FIG. 1F. Graphs illustrating qRT-PCR analyses of mRNA levels of Wnt/β-catenin pathway-target genes and chondrogenic differentiation markers in ATDC5 cells cultured in alginate beads with 50 ng/ml recombinant WNT3A for 48 hours after transfection with pEGFP-N1 or GFP-CXXC5 (mean±s.e.m., n=3, *P<0.05 and **P<0.005).

FIG. 2A. Immunoblotting (upper) and quantitative analyses (mean±s.e.m., n=3) (lower) illustrating the expression of indicated proteins in C28/I2 cells treated with 100 nM E₂(17β-estradiol) for 0, 1, 3, 6, 24.48, or 72 hours.

FIG. 2B. Immunocytochemical staining (left) and quantitative analyses of the fluorescent intensity (mean±s.e.m., n=3, **P<0.005 and ***P<0.0005) (right) illustrating the expression of indicated proteins in C28/I2 cells treated with 100 nM E₂ for 40 hours. Scale bars, 100 μm.

FIG. 2C. Representative images at 6 days (left) and quantitative analyses of the growth changes (mean±s.e.m., n=5, ***P<0.0005) (right). Scale bar, 1 mm. Tibial organ cultures (E15.5) incubated with 100 nM E₂ for 6 days.

FIG. 2D. Hematoxylin and eosin (H&E) staining (left) and quantification of each zone height (mean±s.e.m., n=5, *P<0.05 and **P<0.005) (right) in the growth plate. RZ, resting zone; PZ, proliferative zone; HZ, hypertrophic zone. Scale bar, 200 μm. Tibial organ cultures (E15.5) incubated with 100 nM E₂ for 6 days.

FIG. 2E. Immunohistochemical analyses of β-catenin and CXXCS. Scale bar, 50 μm. Tibial organ cultures (E15.5) incubated with 100 nM E₂ for 6 days.

FIG. 2F. Representative images of H&E staining and immunohistochemical analyses for BrdU, β-catenin, and CXXC5 l in the growth plates of proximal tibiae. 3-week-old Cxxc5^{+/+} and Cxxc5^{-/-} mice were treated with E₂ cypionate (70 μg/kg) by intramuscular (i.m.) injection once a week for 3 weeks (n=3˜4). The area within the dashed lines indicates the growth plate zone. Scale bars, 50 μm.

FIG. 3A. Representative radiographs of tibiae of 12-week-old Cxxc5^{+/+} and Cxxc5^{-/-} mice.

FIG. 3B. Graph illustrating tibial length of 3-, 6-, 9-, and 12-week-old Cxxc5^{+/+} and Cxxc5^{-/-} mice (mean±s.e.m., n=4-10 mice per group, ***P<0.0005).

FIG. 3C. H&E staining in the growth plate of proximal tibiae of 3-, 6-, 9-, and 12-week-old Cxxc5^{+/+} and Cxxc5^{-/-} mice.

FIG. 3D. Graphs illustrating quantitative analyses of each zone height in the growth plate of proximal tibiae of 3-, 6-, 9-, and 12-week-old Cxxc5^{+/+} and Cxxc5^{-/-} mice (mean±s.e.m., n=5, *P<0.05, **P<0.005, and ***P<0.0005).

FIG. 3E. Graph illustrating quantitative analyses of the cell number per column in the growth plates of 9- and 12-week-old Cxxc5^{+/+} and Cxxc5^{-/-} mice (mean±s.e.m., n=5, **P<0.005).

FIG. 3F. Immunohistochemical analyses illustrating the expression of the indicated proteins or in situ hybridization for Runx2 in the proximal tibial growth plates of 11-week-old Cxxc5^{+/+} and Cxxc5^{-/-} mice.

FIG. 3G. Graphs illustrating qRT-PCR analyses of mRNA levels of Wnt-target genes and chondrogenic markers in the growth plate of proximal tibiae of 9-week-old Cxxc5^{+/+} and Cxxc5^{-/-} mice (mean±s.e.m., n=3, *P<0.05, **P<0.005, and ***P<0.0005).

FIG. 3H. in vivo fluorescent imaging shows the presence of the PTD-DBMP in the treated mice (H). The PTD-DBMP (1 mg/kg) were administered to 7-week-old mice by daily intraperitoneal (i.p.) injection for 2 weeks (n=3). White arrowheads indicate the growth plate regions of tibia.

FIG. 3I. H&E staining illustrating immunohistochemical analyses for β-catenin and RUNX2 in the growth plates of proximal tibiae.

FIG. 3J. Graph illustrating quantitative analyses of the cell number in the RZ, PZ, and. HZ of growth plates (mean±s.e.m., n=3, *P<0.05 and **P<0.005). Scale bars, 50 μm.

FIG. 4A. Chemical structure of KY19382.

FIG. 4B. Graph illustrating in vitro binding assay to analyze the effect of KY19382 on CXXCS-DVL interaction (mean±s.e.m., n=3). The IC₅₀ value was determined from the dose-response curve.

FIG. 4C. Graph illustrating in vitro kinase assay to analyze the effect of KY19382 on kinase activity of GSK3β (mean±s.e.m., n=3). The IC₅₀ value was determined from the dose-response curve.

FIG. 4D. Graph illustrating analyses of TOPFlash activity in HEK293 reporter cells grown with the indicated concentrations of KY19382 for 18 hours (mean±s.e.m., n=4, **P<0.005 and ***P<0.0005 versus DMSO-treated control).

FIG. 4E. Immunoblot analyses illustrating the expression of the indicated proteins in ATDC5 cells treated with I3O or KY19382 for 24 hours.

FIG. 4F. Immunoblot analyses of whole cell lysates immunoprecipitated with anti-Mvc in ATDC5 cells treated with 0.1 μM KY19382 for 4 hours after transfection with pCMV-FLAG-DVL1 and pcDNA3.1-CXXC5-Myc.

FIG. 4G. Immunocytochemical staining (left) and quantitative analyses (mean±s.e.m., n=3, **P<0.005) (right) illustrating the effect on β-catenin levels in ATDC5 cells treated with 0.1 μM KY for 48 hours. Scale bar, 100 μm.

FIG. 5A. H&E staining illustrating immunohistochemical analyses with the indicated antibodies. KY19382 (0.1 mg/kg) was administered to 7-week-old mice by daily intraperitoneal injection for 2 weeks (n=7). Scale bars, 50 μm.

FIG. 5B. Graph illustrating quantitative analyses of the cell number per column (mean±s.e.m., n=7, ***P<0.0005) of resting zone and proliferative zone (RZ&PZ) and hypertrophic zone (HZ) in the growth plates of proximal tibiae. KY19382 (0.1 mg/kg) was administered to 7-week-old mice by daily intraperitoneal injection for 2 weeks (n=7).

FIG. 5C. Graph illustrating quantitative analyses of BrdU-positive cells in the growth plates (mean±s.e.m., n=5, ***P<0.0005). KY19382 (0.1 mg/kg) was administered to 7-week-old mice by daily intraperitoneal injection for 2 weeks (n=7).

FIG. 5D. Graph illustrating quantitative analyses of the number of TRAP-positive foci along 250 μm of the cartilage/bone interface (mean±s.e.m., n=3, *P<0.05). KY19382 (0.1 mg/kg) was administered to 7-week-old mice by daily intraperitoneal injection for 2 weeks (n=7).

FIG. 5E. Immunoblot analyses illustrating the expression of the indicated proteins in the growth plate of proximal tibiae of mice treated with KY19382. KY19382 (0.1 mg/kg) was administered to 7-week-old mice by daily intraperitoneal injection for 2 weeks (n=7).

FIG. 5F. TRAP staining in the growth plates of proximal tibiae treated with KY19382 (A, F). TRAP, tartrate-resistant acid phosphatase. KY19382 (0.1 mg/kg) was administered to 3-week-old mice by daily intraperitoneal injection for 2 weeks (n=7). Scale bars, 50 μm.

FIG. 5G. Graph illustrating quantitative analyses of the height (mean±s.e.m., n=7, ***P<0.0005) of resting zone and proliferative zone (RZ&PZ) and hypertrophic zone (HZ) in the growth plates of proximal tibiae. KY19382 (0.1 mg/kg) was administered to 3-week-old mice by daily intraperitoneal injection for 2 weeks (n=7).

FIG. 5H. Graph illustrating quantitative analyses of BrdU-positive cells in the growth plates (mean±s.e.m., n=5, ***P<0.0005). KY19382 (0.1 mg/kg) was administered to 3-week-old mice by daily intraperitoneal injection for 2 weeks (n=7).

FIG. 5I Graph illustrating quantitative analyses of the number of TRAP-positive foci along 250 μm of the cartilage/bone interface (mean±s.e.m., n=3, *P<0.05). KY19382 (0.1 mg/kg) was administered to 3-week-old mice by daily intraperitoneal injection for 2 weeks (n=7). n.s., no significance.

FIG. 5J. Representative radiographs are shown (left), and tibial length was measured (right) (mean±s.e.m., n=7˜15, ***P<0.0005). The area within the dashed lines indicates the growth plate zone. 3-week-old mice were intraperitoneally injected with KY19382 (0.1 mg/kg) daily for 10 weeks.

FIG. 6A. Schematic diagram illustrating a proposed model for the role of CXXC5 l in the growth plate senescence. With pubertal progression, estrogen, which increases during sexual maturation, induces CXXC5 l expression and subsequently inhibits the Wnt/β-catenin pathway via DVL binding, resulting in growth plate senescence.

FIG. 6B. Schematic diagram illustrating a working model of KY19382 for the stimulation of longitudinal bone growth. In activating Wnt/β-catenin signaling, KY19382 functions as a dual-targeting compound by 1) inactivating GSK3β and 2) inhibiting CXXC5-DVL interaction, which results in the delaying of growth plate senescence and the promotion of longitudinal bone growth. PPI, Protein—protein interaction.

FIG. 7A. Graph illustrating analyses of the relative mRNA expression of CXXC5 and CXXC4 in growth plates of human during the pubertal period from microarray data (GEO: GSE9160) (mean±s.e.m., n=2, *P<0.05 and **P<0.005)

FIG. 7B. Graph illustrating analyses of the relative mRNA expression of CXXC5 and CXXC4 in growth plates of rat during the pubertal period from microarray data (GEO: GSE16981) (mean±s.e.m., n=5, ***P<0.0005).

FIG. 8. Graph showing the screening results of small molecules. An in vitro binding assay was performed for 2,280 compounds (30 μM) to identify inhibitors of the CXXC5-DVL interaction. The binding values were calculated by percent ratio of fluorescent intensity normalized to the DMSO-treated control.

FIG. 9A. Diagram illustrating the binding mode of BIO or I3O docked on DVL PDZ (PDB: 2KAW) is shown as a stick model. Structural simulation of the BIO-DVL PDZ complex showed that residues F261, I262, I264, I266, L321, and V325 are involved in binding with BIO: nonbonded interactions (F261, I262, I266, L321, and V325) and hydrogen bonds (I264).

FIG. 9B. Diagram illustrating structural simulation of the I3O-DVL PDZ complex revealed that residues H260, I262, and V325 are involved in binding with I3O: non-bonded interactions (I262 and V325) and hydrogen bonds (H260) (B). BE, Binding Energy.

FIG. 10A. Schematic diagram illustrating Focused Design of indirubin derivatives for the activation of Wnt/β-catenin signaling was directed by modifications of the functional group at the R1 and R2 sites of the indirubin backbone. The synthesized derivatives were analyzed by three assays: (1) in vitro CXXCS-DVL binding assay, (2) in vitro GSK3β kinase assay, and (3) TOPFlash Wnt reporter assay.

FIG. 10B. Diagram illustrating Binding mode of KY19382 docked on DVL PDZ (PDB: 2KAW) is shown as a stick model (left). In structure-based pharmacophore features of KY19382-DVL PDZ (cyan, hydrophobe; green, hydrogen bond acceptor; purple, hydrogen bond donor), the electrostatic surface of DVL PDZ is shown as blue, positively charged; red, negatively charged; white, neural resides (right). The model showed that DVL PDZ residues G263, I264, I266, L321, R322, and V325 are involved in binding with KY19382: non-bonded interactions (I266, L321, R322, and V325) and hydrogen bonds (G263, I264 and V325). BE, Binding Energy.

FIG. 11A. Immunofluorescent staining illustrating the effects of KY19382 on chondrocyte proliferation and differentiation. ATDC5 cells treated with 0.01 or 0.1 μM concentrations of KY19382 were incubated for 48 hours followed by treatment with 50 μM BrdU for 12 hours prior to harvesting. BrdU incorporation was visualized by immunofluorescent staining using a specific BrdU antibody (left). BrdU-positive cells were quantified (mean±s.e.m., n=3, *P<0.05 and ***P<0.0005) (right). Scale bar, 100 μm.

FIG. 11B. Graphs illustrating qRT-PCR analyses of mRNA levels of chondrogenic differentiation markers in ATDC5 cells incubated with 0.1 μM KY19382 for 3 days in three-dimensional alginate beads after transfection with control siRNA or Ctnnb1 siRNA (mean±s.e.m., n=3, *P<0.05, **P<0.005, and ***P<0.0005).

FIG. 11C. Graphs illustrating qRT-PCR analyses of mRNA levels of chondrogenic differentiation markers in C28/I2 cells incubated with 1 μM KY19382 for 3 days in three-dimensional alginate beads (mean±s.e.m., n=3, *P<0.05 and ***P<0.0005).

FIG. 12. Graphs illustrating qRT-PCR analyses of mRNA levels of pathway-specific target genes in ATDC5 cells treated with 0.01, or 0.1 μM concentrations of KY19382 for 4 hours (mean±s.e.m., n=3, *P<0.05 and **P<0.005 versus DMSO-treated control). n.s., no significance versus DMSO-treated control.

FIG. 13A. H&E staining illustrating the effects of KY19382 on articular cartilage. Scale bars, 100 μm.

FIG. 13B. H&E staining illustrating the effects of KY19382 on liver tissues. Scale bars, 100 μm.

FIG. 13C. Graph illustrating the effects of KY19382 on weight. During treatment, weight of mice was measured every 5 to 7 days (mean±s.e.m., n=7˜15). n.s., no significance versus vehicle (control).

FIG. 14. Chemical structures of examples of indirubin analogs disclosed herein.

FIG. 15. Chemical structures of examples of indirubin analogs disclosed herein.

FIG. 16. Chemical structures of examples of indirubin analogs disclosed herein.

BRIEF DESCRIPTION OF SEQUENCES
SEQ ID NO. 1
AUUACAAUCCGGUUGUGAACGUCCC.
SEQ ID NO. 2
GGGACGUUCACAACCGGAUUGUAAU.
SEQ ID NO. 3
UAAUGAAGGCGAACGGCAUUCUGGG.
SEQ ID NO. 4
CCCAGAAUGCCGUUCGCCUUCAUUA.
SEQ ID NO. 5
AGAGCTACGAGCTGCCTGAC.
SEQ ID NO. 6
AGCACTGTGTTGGCGTACA.
SEQ ID NO. 7
TGGAAAGCCTGGTGATGATGGTG.
SEQ ID NO. 8
TGACCTTTGACACCAGGAAGGC.
SEQ ID NO. 9
GAAGACCTCCAGTTTGCAGAGC.
SEQ ID NO. 10
TTCAGGATTCCCGCGAGATTTG.
SEQ ID NO. 11
CACCTTGACCATAACCGTCTTCAC.
SEQ ID NO. 12
CATCAAGCTTCTGTCTGTGCCTTC.
SEQ ID NO. 13
AGGGCAGAATCATCACGAAGTGG.
SEQ ID NO. 14
GTCTCGATTGGATGGCAGTAGC.
SEQ ID NO. 15
GGATGCAGAAGGAGATTACT.
SEQ ID NO. 16
CCGATCCCACACAGAGTACTT.
SEQ ID NO. 17
GGGACTGGTACTCGGATAAC.
SEQ ID NO. 18
CTGATATGCGATGTCCTTGC.
SEQ ID NO. 19
GCCTGTCTGCTTCTTGTAA.
SEQ ID NO. 20
TGCGGTTGGAAAGTGTTT.
SEQ ID NO. 21
TCCACTCGTCCTTCTCAG.
SEQ ID NO. 22
TTTAGCCTACCTCCAAATGC.
SEQ ID NO. 23
ACAAGCCACAAGATTACAAGAA.
SEQ ID NO. 24
GCACCAATATCAAGTCCAAGA
SEQ ID NO. 25
ACCCAGAAGACTGTGGATGG.
SEQ ID NO. 26
GGATGCAGGGATGATGTTCT.
SEQ ID NO. 27
TGAAGTCTCAGAAGGTGGAT.
SEQ ID NO. 28
ATGGCAGAAATAGGCTTTGT.
SEQ ID NO. 29
TAAGACACAGCAAGCCAGA.
SEQ ID NO. 30
CACATCAGTAAGCACCAAGT.
SEQ ID NO. 31
AAGGACAGAGTCAGATTACAGA.
SEQ ID NO. 32
GTGGTGGAGTGGATGGAT.
SEQ ID NO. 33
AACTGGAAACCTGTCTCTCT.
SEQ ID NO. 34
ACAACACACGCACACATC.
SEQ ID NO. 35
TTATTTATTGGTGCTACTGTTTATCC.
SEQ ID NO. 36
TCTGTATTTCTTTGTTGCTGTTT.
SEQ ID NO. 37
YRRAAVPPSPSLSRHSSPHQS(p)EDEEE.

### Definitions

“About” refers to a range of values plus or minus 10 percent, e.g. about 1.0 encompasses values from 0.9 to 1.1.

“CXXC5-DVL interface” refers to an interaction and/or association between CXXC5(CXXC finger protein 5) and DVL (dishevelled), which can induce biological activities known in the art. The interactions and/or associations can be physical or chemical interactions that would activate a CXXC5-DVL pathway within a subject. CXXC5-DVL interface can be present in a form of a complex.

“Growth-related disease or a similar condition” can include, but is not limited to, a growth disorder, human growth hormone deficiency, Cushing's Syndrome, hypothyroidism, nutritional short stature, intrauterine growth retardation, Russell Silver syndrome, disproportionate short stature, achondroplasia, a growth-related disorder, a poor nutrition and systemic disease, a bone disorder, and/or precocious puberty.

“Inhibitor of CXXC5-DVL interface” refers to an agent that alters the function and/or activity of the CXXC5-DVL interface or induces conformational changes in the CXXC5-DVL interface. Examples of inhibitors of CXXC5-DVL interface include, but are not limited to, agents that alter association/dissociation between CXXC5 and DVL and/or agents that inhibit CXXC5-DVL complex assembly/function.

“Pharmaceutically acceptable” refers to approved or approvable by a regulatory agency of a government, such as the U.S. FDA or the EMA, or listed in the U.S. Pharmacopoeia or other generally recognized pharmacopoeia for use in mammals and/or animals, and more particularly in humans.

“Pharmaceutically acceptable vehicle” or “pharmaceutically acceptable carrier,” unless stated or implied otherwise, is used herein to describe any ingredient other than the active component(s) that can be included in a formulation. The choice of carrier will to a large extent depend on factors such as the mode of administration, the effect of the carrier on solubility and stability, and the nature of the dosage form.

“Pharmaceutical composition” refers to a therapeutically active inhibitor of CXXC5-DVL interface or a therapeutically active inhibitor of GSKβ, and at least one pharmaceutically acceptable vehicle/carrier, with which the inhibitor of CXXC5-DVL interface and/or inhibitor of GSKβ is administered to a subject.

“Subject” refers to a human (adult and/or child), an animal, a livestock, a cell, and/or a tissue.

“Therapeutically effective amount” refers to the amount of an inhibitor of CXXC5-DVL interface or inhibitor of GSKβ that, when administered to a subject for treating a disease, or at least one of the clinical symptoms of a disease, is sufficient to affect such treatment of the disease or symptom thereof The “therapeutically effective amount” can vary depending, for example, on the inhibitor of CXXC5-DVL interface, inhibitor of GSKβ, the disease and/or symptoms of the disease, severity of the disease and/or symptoms of the disease or disorder, the age, weight, and/or health of the subject to be treated, and the judgment of the prescribing physician.

“Therapeutically effective dose” refers to a dose that provides effective treatment of a disease or disorder in a subject. A therapeutically effective dose can vary from compound to compound, and from subject to subject, and can depend upon factors such as the condition of the subject and the route of delivery.

“Therapeutic regime(s)” and/or “therapeutic regimen(s)” include, but are not limited to, growth hormone therapy, sex hormone therapy, and/or surgery.

“Treat,” “treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. In some embodiments, “treat,” “treating” or “treatment” also refers to inhibiting the disease, either physically, (e.g., stabilization of a discernible symptom), physiologically, (e.g., stabilization of a physical parameter), or both, and to inhibiting at least one physical parameter that can or cannot be discernible to the subject. In certain embodiments, “treat,” “treating” or “treatment” refers to delaying the onset of the disease or at least one or more symptoms thereof in a subject which can be exposed to or predisposed to a disease even though that subject does not yet experience or display symptoms of the disease.

### DETAILED DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

Longitudinal bone growth occurs rapidly during fetal development and early childhood, but then it gradually slows down and eventually ceases at the end of puberty with growth plate senescence.

The mechanisms for regulating growth plate senescence are not well understood. In recent years, accumulating evidence from basic and clinical studies revealed that chondrocyte activity and status is directly subject to regulation by paracrine signaling within the growth plate. Further, Wnt/β-catenin signaling has emerged as a key player in growth plate maturation, and mutation of genes involved in the regulation of Wnt/β-catenin signaling often resulted in impaired bone growth. For example, cartilage-specific loss of Ctnnb1 encoding β-catenin caused defects in longitudinal bone growth. Additionally, treatment with an inhibitor of glycogen synthase kinase 3β (GSK3β), a serine/threonine kinase that destabilizes β-catenin, resulted in tibial elongation in the ex-vivo culture system. Furthermore, a large meta-analysis of genome-wide association studies identified 423 loci that contribute to common variation in adult human height and found genes involved in the Wnt/β-catenin pathway such as AXIN2, WNT4, and CTNNB1.

CXXC finger protein 5 (CXXC5) is a negative regulator of Wnt/β-catenin signaling, functioning via interaction with PDZ domain of dishevelled (DVL) in the cytosol. Inhibition of the CXXC5-DVL interaction improved several pathophysiological phenotypes involving Wnt/β-catenin signaling including osteoporosis, cutaneous wounds, and hair loss through activation of the Wnt/β-catenin signaling.

In this disclosure, CXXC5 expression was found to be progressively increased in chondrocytes undergoing growth plate senescence. Further, estrogen, a sex hormone that is elevated during the pubertal period, induced CXXC5 expression followed by decrement of β-catenin in chondrocytes. Cxxc5^{-/-} mice displayed enhanced chondrocyte proliferation and differentiation in the late pubertal growth plate as well as longer tibiae at adulthood. The results disclosed herein suggest that CXXC5 contributes to growth plate senescence at puberty. Thus, the instant disclosure provides a novel function of the CXXC5-DVL interface that may lead to downregulation of bone growth in a subject.

The present disclosure provides, inter alia, a discovery platform for developing therapeutic inhibitors of the CXXC5-DVL interface that is thought to negatively affect the Wnt/β-catenin pathway, for example, in chondrocytes undergoing growth plate senescence. In some embodiments, small molecules that activate the Wnt/β-catenin pathway by inhibiting the CXXC5-DVL interface are obtained by use of an in vitro screening system monitoring fluorescent intensity that reveals binding of the PTD-DBMP (protein transduction domain fused DVL binding motif peptide), which contains sequence of CXXC5 binding to DVL and is conjugated to FITC, onto PZD domain of DVL. See e.g., Kim H Y, et al (2016), Small molecule inhibitors of the Dishevelled-CXXC5 interaction are new drug candidates for bone anabolic osteoporosis therapy. EMBO Mol Med 8: 375-387. Interestingly, several GSK3β inhibitors, including 6-bromoindirubine-3′-oxime (BIO) and indirubin 3′-oxyme (I3O), were identified as initial hits. Further, the instant disclosure provides that a functionally improved, and newly synthesized, indirubin derivative, KY19382, effectively inhibited both GSK3β kinase activity and CXXCS-DVL interaction. These functions were confirmed by kinetic measurement of GSK3β enzyme activity and in vitro CXXC5-DVL binding, respectively. Therefore, KY19382 effectively activated Wnt/β-catenin signaling via at least one of the functions: (1) initial activation by inhibition of GSK3β and/or (2) enhancement of the signaling by interference of CXXC5-DVL interaction. Further, KY19382 markedly enhanced proliferation and differentiation of chondrocytes and induced longitudinal tibiae growth in adolescent mice by delaying growth plate senescence. By identifying this CXXC5-DVL induced mechanism of growth plate senescence, the present disclosure provides a platform for screening compound libraries for inhibitors of the specific interaction of CXXC5 and DVL by binding to the CXXCS-DVL interface that involves the DVL binding motif.

Embodiments disclosed herein relate to compositions and methods for treating a condition and/or disease associated with growth or a related clinical condition in a subject. In certain embodiments, compositions and methods disclosed herein concern suppression of a side effect of a therapeutic regime. Other embodiments relate to compositions and methods for treating a subject diagnosed with a growth related disease or having a condition contributed to a growth disorder, human growth hormone deficiency, Cushing's Syndrome, hypothyroidism, nutritional short stature, intrauterine growth retardation, Russell Silver syndrome, disproportionate short stature, achondroplasia, a growth-related disorder, a poor nutrition and systemic disease, a bone disorder, and/or precocious puberty.

Methods disclosed herein include a method of treating a clinical condition, comprising administering to a subject at least one therapeutically effective dose of any one of the compounds and/or compositions disclosed herein. The subject can be diagnosed with a clinical condition selected from and/or comprising a growth-related disease or a similar condition thereof In certain embodiments, the methods disclosed herein further comprise administering to the subject at plurality of therapeutically effective doses of any one of the compounds and/or compositions disclosed herein.

In some embodiments, compositions disclosed herein comprise at least one agent that inhibits CXXC5-DVL interface in a subject. Consistent with these embodiments, the at least one agent that inhibits or reduces the CXXC5-DVL interface comprises at least one compound disclosed herein. In some embodiments, the at least one agent that inhibits or reduces the CXXC5-DVL interface can disrupt conformation of the CXXC5-DVL interface physically and/or chemically.

### Pharmaceutical Compositions

Pharmaceutical compositions provided by the present disclosure can comprise a therapeutically effective amount of one or more compositions disclosed herein, together with a suitable amount of one or more pharmaceutically acceptable vehicles to provide a composition for proper administration to a subject. Suitable pharmaceutical vehicles are described in the art.

Pharmaceutical compositions of the present disclosure suitable for oral administration can be presented as discrete units, such as a capsule, cachet, tablet, or lozenge, each containing a predetermined amount of the active ingredient; as a powder or granules; as a solution or a suspension in an aqueous liquid or non-aqueous liquid such as a syrup, elixir or a draught, or as an oil-in-water liquid emulsion or a water-in-oil liquid emulsion. The composition can also be presented as a bolus, electuary or paste. A tablet can be made by compressing or moulding the active ingredient with the pharmaceutically acceptable carrier. Compressed tablets can be prepared by compressing in a suitable machine the active ingredient in a free-flowing form, such as a powder or granules, in admixture with, for example, a binding agent, an inert diluent, a lubricating agent, a disintegrating and/or a surface-active agent. Moulded tablets can be prepared by moulding in a suitable machine a mixture of the powdered active ingredient moistened with an inert liquid diluent. The tablets can optionally be coated or scored and can be formulated to provide slow or controlled release of the active ingredient.

Pharmaceutical compositions of the present disclosure suitable for parenteral administration include aqueous and non-aqueous sterile injection solutions, and can also include an antioxidant, buffer, a bacteriostat and a solution which renders the composition isotonic with the blood of the recipient, and aqueous and non-aqueous sterile suspensions which can contain, for example, a suspending agent and a thickening agent. The formulations can be presented in a single unit-dose or multi-dose containers and can be stored in a lyophilized condition requiring the addition of a sterile liquid carrier prior to use.

Pharmaceutically acceptable salts include salts of compounds provided by the present disclosure that are safe and effective for use in mammals and that possess a desired therapeutic activity. Pharmaceutically acceptable salts include salts of acidic or basic groups present in compounds provided by the present disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain disclosed compounds may form pharmaceutically acceptable salts with various amino acids. Suitable base salts include, but are not limited to, aluminum, calcium, lithium, magnesium, potassium, sodium, zinc, and diethanolamine salts. For additional information on some pharmaceutically acceptable salts that can be used to practice the methods described herein please review articles such as Berge, et al., 66 J. PHARM. SCI. 1-19 (1977), Haynes, et al, J. Pharma. Sci., Vol. 94, No. 10, October 2005, pgs. 2111-2120, and the like.

In some embodiments, the composition can contain pharmaceutically acceptable lubricant(s). The pharmaceutically acceptable lubricant(s) prevent the components of the pharmaceutical composition from clumping together and from sticking to the pellet press that generates the disclosed compositions. The lubricant(s) also ensure that the formation of the pellet, as well as its ejection from the pellet press, occurs with low friction between the composition and the wall of the die press. In some embodiments, the lubricant(s) are added to a pharmaceutical composition to improve processing characteristics, for example to help increase the flexibility of the compositions, thereby reducing breakage.

The type of lubricant that can be used in the disclosed pharmaceutical compositions can vary. In some embodiments, the pharmaceutically acceptable lubricant is selected from talc, silica, vegetable stearin, magnesium stearate, stearic acid, calcium stearate, glyceryl behenate, glyceryl monostearate, glyceryl palmitostearate, mineral oil, polyethylene glycol, sodium stearyl fumarate, sodium lauryl sulfate, vegetable oil, zinc stearate, and combinations thereof. In some embodiments, the pharmaceutically acceptable lubricant is stearic acid.

The type of vehicles that can be used in the disclosed pharmaceutical compositions can vary. In some embodiments, the pharmaceutically acceptable vehicles are selected from binders, fillers and combinations thereof. In some embodiments, the pharmaceutically acceptable vehicle is selected from ascorbic acid, polyvinylpyrrolidone, polyvinylpyrrolidone K-30 (povidone K-30), glyceryl monostearate (GMS) or glyceryl monostearate salts, glyceryl behenate, glyceryl palmitostearate, hydroxypropyl cellulose, hydroxypropyl methylcellulose, methylcellulose, hydroxyethyl cellulose, sugars, dextran, cornstarch, dibasic calcium phosphate, dibasic calcium phosphate dihydrate, calcium sulfate, dicalcium phosphate, tricalcium phosphate, lactose, cellulose including microcrystalline cellulose, mannitol, sodium chloride, dry starch, pregelatinized starch, compressible sugar, mannitol, lactose monohydrate, starch, dibasic calcium phosphate dihydrate, calcium sulfate, dicalcium phosphate, tricalcium phosphate, powdered cellulose, microcrystalline cellulose, lactose, glucose, fructose, sucrose, mannose, dextrose, galactose, the corresponding sugar alcohols and other sugar alcohols, such as mannitol, sorbitol, xylitol, and combinations of any of the foregoing. In some embodiments, the pharmaceutically acceptable vehicle is polyvinylpyrrolidone K-30, also known as povidone K-30. In some embodiments, the pharmaceutically acceptable vehicle is polyvinylpyrrolidone K-30, also known as povidone K-30, having an average molecular weight of MW of 40,000 (CAS 9003-39-8). In some embodiments, the pharmaceutically acceptable vehicle is selected from glyceryl monostearate (GMS) or glyceryl monostearate salts, glyceryl behenate and glyceryl palmitostearate. In some embodiments, the pharmaceutically acceptable vehicle is glyceryl monostearate (GMS) or glyceryl monostearate salts. In some embodiments, the pharmaceutically acceptable vehicle is glyceryl behenate. In some embodiments, the pharmaceutically acceptable vehicle is glyceryl palmitostearate.

In some embodiments, the antioxidants prevent oxidation of the other components of the disclosed compositions. Oxidation can occur, for example, during sterilization where free radicals are generated. Addition of the antioxidants, or free radical scavengers, significantly reduces oxidation and makes the composition more pharmaceutically acceptable for use in subjects.

The type of antioxidants that can be used in the disclosed pharmaceutical compositions can vary. In some embodiments, the antioxidant is selected from methyl paraben and salts thereof, propyl paraben and salts thereof, vitamin E, vitamin E TPGS, propyl gallate, sulfites, ascorbic acid (aka L-ascorbic acid, also including the L-enantiomer of ascorbic acid, vitamin C), sodium benzoate, citric acid, cyclodextrins, peroxide scavengers, benzoic acid, ethylenediaminetetraacetic acid (EDTA) and salts thereof, chain terminators (e.g., thiols and phenols), butylated hydroxytoluene (BHT), butylated hydroxyanisole (BHA), and combinations thereof.

### Uses or Methods of Treatment

The methods and compositions disclosed herein can be used to treat subjects suffering from diseases, disorders, conditions, and symptoms for which inhibitors of the CXXC5-DVL interface and/or GSKβ are known to provide or are later found to provide therapeutic benefit.

In some embodiments, methods disclosed herein include a method of treating a clinical condition, comprising administering to a subject at least one therapeutically effective dose of any of the compositions disclosed herein. The subject can be diagnosed with a clinical condition selected from and/or comprising growth disorders, human growth hormone deficiency, Cushing's Syndrome, hypothyroidism, nutritional short stature, intrauterine growth retardation, Russell Silver syndrome, disproportionate short stature, achondroplasia, growth related disorders, poor nutrition and systemic diseases, bone disorders, and/or precocious puberty, and/or any other conditions associated with, induced by, or that are already resistant to growth hormone therapy and/or surgical treatments. In certain embodiments, the methods disclosed herein further comprise administering to the subject at least one additional therapeutically effective dose of any of the compositions disclosed herein. In some embodiments, the at least one therapeutically effective dose of any of the compositions disclosed herein can be administered orally, parenterally, intravenously, by inhalation and/or transdermally.

Yet other embodiments can include methods for reducing a side effect of a therapeutic regime, comprising administering to a subject at least one therapeutically effective dose of at least one agent that inhibits or reduces the activity of the CXXC5-DVL interface in a subject; wherein the subject has received at least one therapeutic regime comprising surgery, growth and/or sex hormone therapy, and wherein the subject experiences at least one side effect derived from the therapeutic regime. Consistent with these embodiments, side effects can include, but are not limited to, drug-resistance and/or relapse.

### Kits

In a further aspect, kits are provided by the present disclosure, such kits comprising: one or more pharmaceutical compositions, each composition sterilized within a container, means for administration of the pharmaceutical compositions to a subject, and instructions for use.

Some embodiments include kits for carrying out the methods disclosed herein. Such kits typically comprise two or more components required for treating a clinical condition. Components of the kit include, but are not limited to, one or more of agents/compositions disclosed herein, reagents, containers, equipment and/or instructions for using the kit. Accordingly, the compositions and methods described herein can be performed by utilizing pre-packaged kits disclosed herein.

### EXAMPLES

The following examples illustrate various aspects of the disclosure. It will be apparent to those skilled in the art that many modifications, both to materials and methods, can be practiced without departing from the scope of the disclosure.

Cell culture and reagents. The mouse chondrogenic cell line, ATDC5, was obtained from the RIKEN Cell Batik. The human juvenile costal chondrocyte cell line, C28/I2, was provided by Dr. W. U. Kim (Catholic University, Korea). HEK293-TOP cells (HEK293 cells containing the chromosomally incorporated TOPFlash gene) were provided by Dr. S. Oh (Kuk Min University, Korea). ATDCS cells were maintained in DMEM/F12 (1:1) (Gibco, Grand Island, N.Y.) supplemented with 5% FBS (Gibco). To induce hypertrophic differentiation, ATDC5 cells were incubated with insulin-transferrin-sodium selenite (ITS) supplement (Gibco) in three-dimensional alginate beads for 3 days, as previously described. See e.g., Kawasaki Y. et al. (2008), Phosphorylation of GSK-3beta by cGMP-dependent protein kinase II promotes hypertrophic differentiation of murine chondrocytes. J CLIN INVEST 118: 2506-2515. C28/I2 and HEK293-TOP cells were maintained in DMEM (Gibco) containing 10% FBS. All chemicals were dissolved in dimethyl sulfoxide (DMSO; Sigma-Aldrich, St Louis, Mo.) for the in vitro studies. For E₂ (17β-estradiol; Sigma-Aldrich) treatment, cells were cultured in phenol red-free DMEM/F12 with 5% charcoal-stripped FBS for 24 hours followed by serum-free medium for 24 hours before the experiment. The PTD-DBMP was synthesized by Peptide 2.0 Inc (Chantilly, Va.).

Plasmids, siRNAs, and transfection. The plasmids pcDNA3.1-CXXC5-Myc and GFP-CXXC5 l have been previously described (Kim et al, 2015). The pCMV-FLAG-DVL1 was provided by Dr. E. H. Jho (Seoulsirip university, Korea). The following siRNA sequences were used for ATDCS cells: Ctnnb1 (encoding β-catenin) siRNA-1, sense AUUACAAUCCGGUUGUGAACGUCCC (SEQ ID NO. 1) and anti-sense GGGACGUUCACAACCGGAUUGUAAU (SEQ ID NO. 2), Ctnnb1 siRNA-2, sense UAAUGAAGGCGAACGGCAUUCUGGG (SEQ ID NO. 3) and anti-sense CCCAGAAUGCCGUUCGCCUUCAUUA (SEQ ID NO. 4)

Lipofectamine (Invitrogen, Carlsbad, Calif.) was used for plasmid transfection and RNAiMax (Invitrogen) was used for siRNA transfection, according to the manufacturer's instructions.

Animals. Cxxc5^{-/-} mice were established in a previous study. See e.g., Kim H. Y. et al. (2015), CXXC5 is a negative feedback regulator of the Wnt/beta-catenin pathway involved in osteoblast differentiation. CELL DEATH DIFFER 22: 912-920. To manipulate growth plate senescence by estrogen, 3-week-old Cxxc5^{-/-} and Cxxc5^{-/-} male mice received weekly intramuscular (i.m.) injections of either 70 μg/kg estradiol (E₂) cypionate (Sigma-Aldrich) or vehicle (cottonseed oil) for 3 weeks. To investigate the effects of KY19382 treatment on longitudinal bone growth, C57BL/6 male mice were purchased from KOATECH (Gyeonggido, Korea). KY19382 (0.1 ing/kg) was administered daily by intraperitoneal (i.p.) injection to 3- and 7-week-old mice for 2 weeks or to 3-week-old mice for 10 weeks. For BrdU labeling experiments, mice were i.p. injected with 50 mg/kg BrdU (Sigma-Aldrich) before 24 hours to sacrifice. All animal procedures were approved by the Institutional Animal Care and Use Committee (IACUC) of Yonsei University (Korea) and conducted based on the guidelines of the Korean Food and Drug Administration.

Radiographic and histochemical analyses. Plain radiographs were taken using an X-ray apparatus (KODAK DXS 4000 Pro SYSTEM; Carestream Health Rochester, N.Y.). The tissues were fixed in 4% paraformaldehyde (PFA), decalcified in 10% EDTA (pH 7.4), dehydrated, embedded in paraffin, and sectioned to 4 μm thickness (Leica Microsystems, Wetzlar, Germany). The tissues sections were rehydrated and used for further analyses including H&E, TRAP, and itnmunohistochemical (IHC) staining. To perform IHC staining, the sections were incubated with citrate buffer (pH 6.0) at 80° C. for 30 minutes, with 0.05% trypsin working solution (pH 7.8) for 30 minutes at 37° C., or with 0.5% pepsin (Sigma-Aldrich) for 15 minutes at 37° C. Then, the sections were blocked with 5% normal goat serum (NGS; Vector Laboratories, Burlingame, Calif.) and 0.3% triton-x-100 in PBS for 1 hour at room temperature. For 3,3′-diaminobensidine (DAB) staining, the sections were incubated with 0.345% H₂O₂ for 15 minutes. Before incubating the sections with mouse primary antibody, mouse IgG was blocked using a M.O.M kit (Vector Laboratories). The sections were incubated at 4° C. overnight with the following primary antibodies: anti-β-catenin (BD Bioscience, San Jose, Calif., #610154; 1:50), anti-CXXC5 l (1:200; lab-made) anti-BrdU (DAKO, Carpinteria, Calif., M0744; 1:200), anti-COL2A1 (ThermoFisher Scientific, MA, PAS-11462; 1:100), anti-Ki67 (Abcam, Cambridge, UK, ab15580; 1:200), and anti-RUNX2 (Abeam, ab23981; 1:200). Then, the sections were incubated at room temperature for 1 hour with biotinylated anti-mouse (Vector Laboratories, BA-9200; 1:200) or biotinylated anti-rabbit (Vector Laboratories, BA-1000; 1:200) secondary antibodies. The sections were then incubated in avidin-biotin complex solutions (Vector Laboratories), stained with a DAB kit (Vector Laboratories) for 3-30 minutes, and counterstained with methyl green (Sigma-Aldrich). All incubations were conducted in humid chambers. Staining was observed with an ECLIPSE TE2000-U microscope (Nikon, Tokyo, Japan). For fluorescence staining, the sections were incubated with primary antibody at 4° C. overnight, followed by incubation with anti-mouse Alexa Fluor 488 (ThermoFisher Scientific, A11008; 1:200) or anti-rabbit Alex Fluor 555 (ThermoFisher Scientific, A21428; 1:200) secondary antibodies at room temperature for 1 hour. The sections were then counterstained with DAPI (Sigma-Aldrich) for 5 minutes and mounted in Gel/Mount media (Biomeda Corporation). All incubations were conducted in dark humid chambers. The fluorescent signals were visualized using a LSM700 META confocal microscope (Carl Zeiss Inc., Thornwood, N.Y.) at excitation wavelengths of 488 nm (Alexa Fluor 488), 543 nm (Alexa Fluor 555), and 405 nm (DAPI).

Immunocytochemistry. ATDCS or C28/I2 cells were seeded on glass coverslip in 12-well culture plates. The cells were washed with PBS and fixed with 4% PFA at room temperature for 15 minutes. After permeabilization with 0.1% triton-X-100 for 15 minutes and blocking with 5% BSA for 1 hour, the cells were incubated with primary antibodies specific for β-catenin (1:100) or CXXC5 l (1:200) at 4° C. overnight. The cells were washed in PBS and incubated with Alexa Fluor 488 or Alexa Fluor 555 secondary antibodies (1:200) at room temperature for 1 hour. Cell nuclei were counterstained with DAPI for 10 minutes and the stained samples were examined under a LSM700 META microscope using 405-, 488-, or 543-nm excitation wavelengths. For BrdU assay, cultured cells were incubated with BrdU solution (25 μM) overnight, followed by immunocytochemical staining with antibody against BrdU (1:100).

Immunoblot analyses. Cells were washed with ice-cold PBS and tissues were ground with a mortar and pestle in liquid nitrogen before lysis in RIPA buffer (150 mM NaCl, 50 mM Tris, pH 7.4, 1% NP-40, 0.25% sodium deoxycholate, 1 mM EDTA, protease inhibitors, and phosphatase inhibitors). Protein samples were separated on a 8-12% sodium dodecylsulfate polyacrylamide gel electrophoresis (SDS-PAGE) and transferred to a nitrocellulose membrane (Whatman). Immunoblotting was performed with the following primary antibodies: anti-β-catenin (Santa Cruz Biotechnology, Santa Cruz, Calif., sc-7199; 1:3,000), anti-CXXC5 (lab made;1:200) anti-Myc tag (MBL, Aichi, Japan, M192-3; 1:1,000), anti-FLAG (Sigma-Aldrich, F7425; 1:1,000), anti-p-GSK3β (S9; Cell Signaling Technology, Danvers, Mass., 9336S; 1:1,000), anti-COL2A1 (Santa Cruz Biotechnology, sc-28887; 1:500), anti-RUNX2 (Abcam, ab23981; 1:500), anti-COLIOAI (Cosmo Bio, LSL-LB-0092; 1:500), anti-MMP13 (Santa Cruz Biotechnology, sc-30073; 1:500), anti-ERK (Santa Cruz Biotechnology, sc-94; 1:3,000) and anti-α-tubulin (Cell Signaling Technology, 3873S; 1:20,000). Samples were then incubated with horseradish-peroxidase-conjugated anti-mouse (Cell Signaling Technology, 7076; 1:3,000), anti-rabbit (Bio-Rad, 1706515; 1:3,000), or anti-goat (Santa Cruz Biotechnology; sc-2020; 1:3,000) secondary antibodies. Protein bands were visualized with enhanced chemiluminescence (ECL; Amersham Bioscience, Piscataway, N.J.) using a luminescent image analyzer, LAS-3000 (Fujifilm, Tokyo, Japan). Immunoblot bands were analyzed using Multi-Gauge V3.0 software (Fujifilm). Points of interest (POIs) from immunoblot bands were marked and quantified using densitometry, and the background signals were subtracted from respective immunoblot signals. Relative densitometry values were presented as the intensity ratios of each protein to loading control protein (α-tubulin or ERK).

Immunoprecipitation. Immunoprecipitation was performed as previously described (Kim et al, 2015). To monitor the protein-protein interactions, 1 mg of WCLs were incubated with anti-DVL1 and protein G agarose beads (GenDEPOT, Katy, Tex.) or anti-Myc and protein A agarose beads (GenDEPOT) at 4° C. for 16 hours, and the beads were then washed 3 times in RIPA buffer. The resulting immune complexes were resolved by SDS-PAGE, and immunoblotting was performed with the indicated antibodies.

Tibial organ culture. Tibiae were isolated from embryonic day 15.5 (e15.5) mice and cultured for 6 days in phenol red-free a-MEM (Gibco) containing ascorbic acid, β-glycerophosphate, BSA, L-glutamine, and penicillin-streptomycin, as previously described (Gillespie et al, 2011). After dissection, tibiae were incubated in medium overnight and then treated with E₂ (Sigma-Aldrich). Media and reagents were changed every 48 hours. Tibial images were captured using a SMZ-745T microscope (Nikon). Tibial length was measured prior to treatment and after 6 days in culture. The samples were then prepared for paraffin embedding, sectioned, and analyzed by H&E and IHC staining.

Reporter assay. HEK293-TOP cells were seeded into each well of a 24-well plate. The cells were treated with individual compounds at indicated concentration and cultured for 18 hours. The cells were then harvested and lysed in 60 μl of Reporter Lysis Buffer (Promega, Madison, Wis.) according to the manufacturer's instructions. After centrifugation, 20 μl of the supernatant was used to measure luciferase activity. Relative luciferase activities were normalized to that of the DMSO-treated control.

Reverse transcription and quantitative real-time PCR. Total RNA was extracted using Trizol reagent (Invitrogen) according to the manufacturer's instructions. 2 μg of RNA was reverse-transcribed using 200 units of reverse transcriptase (Invitrogen) in a 40-μl reaction carried out at 37° C. for 1 hour. For quantitative real-time PCR analyses (qRT-PCR), the resulting cDNA (1 μl) was amplified in 10 μl reaction mixture containing iQ SYBR. Green Supermix (Qiagen, Germantown, Md.), 10 pmol of the primer set (Bioneer) The comparative cycle-threshold (CT) method was used, and ACTB encoding β-actin or GAPDH served as an endogenous control. The following primer sets were used:

TABLE 1
List of Primers

| Human | | |
|---|---|---|
| Gene | Strand | Primer Sequences |
| ACTB | F | 5′-AGAGCTACGAGCTGCCTG |
| | | AC-3′ SEQ ID NO. 5 |
| | R | 5′-AGCACTGTGTTGGCGTAC |
| | | A-3′ SEQ ID NO. 6 |
| COL2A1 | F | 5′-TGGAAAGCCTGGTGATGA |
| | | TGGTG-3′ SEQ ID NO. 7 |
| | R | 5′-TGACCTTTGACACCAGGA |
| | | AGGC-3′ SEQ ID NO. 8 |
| MMP13 | F | 5′-GAAGACCTCCAGTTTGCA |
| | | CGAG-3′ SEQ ID NO. 9 |
| | R | 5′-TTCAGGATTCCCGCGAGA |
| | | TTTG-3′ SEQ ID NO. 10 |
| RUNX2 | F | 5′-CACCTTGACCATAACCGT |
| | | CTTCAC-3′ SEQ ID NO. 11 |
| | R | 5′-CATCAAGCTTCTGTCTGT |
| | | GCCTTC-3′ SEQ ID NO. 12 |
| VEGFA | F | 5′-AGGGCAGAATCATCACGA |
| | | AGTGG-3′SEQ ID NO. 13 |
| | R | 5′-GTCTCGATTGGATGGCAG |
| | | TAGC-3′ SEQ ID NO. 14 |

TABLE 2
List of Primers

| Mouse | | |
|---|---|---|
| Gene | Strand | Primer Sequences |
| ActB | F | 5′-GGATGCAGAAGGAGATTACT-3′ |
| | | SEQ ID NO. 15 |
| | R | 5′-CCGATCCCACACAGAGTACTT-3′ |
| | | SEQ ID NO. 16 |
| Alp | F | 5′-GGGACTGGTACTCGGATAAC-3′ |
| | | SEQ ID NO. 17 |
| | R | 5′-CTGATATGCGATGTCCTTGC-3′ |
| | | SEQ ID NO. 18 |
| Col2a1 | F | 5′-GCCTGTCTGCTTCTTGTAA-3′ |
| | | SEQ ID NO. 19 |
| | R | 5′-TGCGGTTGGAAAGTGTTT-3′ |
| | | SEQ ID NO. 20 |
| Col10a1 | F | 5′-TCCACTCGTCCTTCTCAG-3′ |
| | | SEQ ID NO. 21 |
| | R | 5′-TTTAGCCTACCTCCAAATGC-3′ |
| | | SEQ ID NO. 22 |
| Ctnnb1 | F | 5′-ACAAGCCACAAGATTACAAGAA-3′ |
| | | SEQ ID NO. 23 |
| | R | 5′-GCACCAATATCAAGTCCAAGA-3′ |
| | | SEQ ID NO. 24 |
| Gapdh | F | 5′-ACCCAGAAGACTGTGGATGG-3′ |
| | | SEQ ID NO. 25 |
| | R | 5′-GGATGCAGGGATGATGTTCT-3′ |
| | | SEQ ID NO. 26 |
| Mmp9 | F | 5′-TGAAGTCTCAGAAGGTGGAT-3′ |
| | | SEQ ID NO. 27 |
| | R | 5′-ATGGCAGAAATAGGCTTTGT-3′ |
| | | SEQ ID NO. 28 |
| Mmp13 | F | 5′-TAAGACACAGCAAGCCAGA-3′ |
| | | SEQ ID NO. 29 |
| | R | 5′-CACATCAGTAAGCACCAAGT-3′ |
| | | SEQ ID NO. 30 |
| Runx2 | F | 5′-AAGGACAGAGTCAGATTACAGA-3′ |
| | | SEQ ID NO. 31 |
| | R | 5′-GTGGTGGAGTGGATGGAT-3′ |
| | | SEQ ID NO. 32 |
| Sox9 | F | 5′-AACTGGAAACCTGTCTCTCT-3′ |
| | | SEQ ID NO. 33 |
| | R | 5′-ACAACACACGCACACATC-3′ |
| | | SEQ ID NO. 34 |
| Vegfa | F | 5′-TTATTTATTGGTGCTACTGTTTATCC-3′ |
| | | SEQ ID NO. 35 |
| | R | 5′-TCTGTATTTCTTTGTTGCTGTTT-3′ |
| | | SEQ ID NO. 36 |

The primer sets of pathway-specific target genes were described in a previous study (Kim et al, 2016).

GSK3β kinase assay. GSK3β (human) was incubated with 8 mM MOPS (pH 7.0), 0.2 mM EDTA, 20 μM YRRAAVPPSPSLSRHSSPHQS(p) EDEEE (phospho-GS2 peptide) (SEQ ID NO. 37), 10 mM Mg acetate, and [γ-33P-ATP] (specific activity approx. 500 cpm/pmol, concentration as required). The reaction was initiated by the addition of the Mg-ATP mixture. After incubation for 40 minutes at room temperature, the reaction was stopped by addition of 3% phosphoric acid solution. 10 μl of the reaction was then spatted onto a P30 filtermat and washed three times for 5 minutes in 50 mM phosphoric acid and once in methanol prior to drying and scintillation counting.

Database. The gene expression profile results were deposited in NCBI's Gene Expression Omnibus database (GEO) (http://www.nchi.nlm.nih.gov/geo/) and are accessible through GEO accession number GSE16981, GSE14007, GSE9160.

Quantitation of signal intensity. For DAB immunostaining, validation of the immnohistochemical scoring (H-score) was performed using the automated digital image analysis software ImageJ (National Institutes of Health, Bethesda, Md.) and the IHC Profiler plug-in (Varghese et al, 2014). For immunofluorescent staining, the intensity was analyzed with NIS Elements V3.2 software (Nikon). The blue channel was used as a reference to visualize the nuclei, and the threshold was defined for red, green, or blue channels. Mean intensity was calculated in the red and green channels separately, and mean values were estimated from analyses of three independent experiments.

Statistical analyses. All data are expressed as the mean±s.e.m., and the number of samples is indicated in each figure legend. The statistical significance of differences was assessed using the unpaired two-tailed Student's t-test. Results shown are representative of at least three independent experiments. Statistical significance is indicated in the figures as follows: * or #P<0.05; ** or ##, P<0.005; *** or ###, P<0.0005.

Screening for compounds that inhibit the CXXC5-DVL interaction. To initially identify small molecules that inhibited the CXXCS-DVL interaction, chemical libraries (2,280 compounds: 1,000 from ChemDiv and 1,280 from SigmaLOPAC) were screened by in vitro binding assay that was previously described (Kim et al, 2016). Briefly, 5 mg/ml purified DVL PDZ domain was incubated in each well of a 96-well Maxibinding Immunoplate (SPL) at 4° C. for 16 hours. After the addition of 10 μM FITC-tagged PTD-DBMP to each well, each compound in the chemical library or control (DMSO) was treated to the well at a final concentration of 30 μM. The fluorescence intensity was measured using a Fluorstar Optima microplate reader (BGM Lab Technologies, Ortenberg, Germany). The binding values were calculated as a percent ratio of the fluorescent intensity normalized to the DMSO-treated control. Nineteen compounds exhibited lower than 10% for the CXXC5-DVL interaction. Among these compounds, indirubin analogs including BIO and I3O were identified. A summary of the high-throughput screening results is provided in Table 3.

TABLE 3
Summary of high-throughput screening results

| Category | Parameter | Description |
|---|---|---|
| Assay | Type of assay | In vitro binding assay |
| | Target | CXXC5-DVL interaction |
| | Primary measurement | Fluorescence intensity |
| | Key reagents | FITC-tagged PTD-DBM |
| | | peptide |
| | Assay protocol | The protocol was |
| | | provided in “Small |
| | | molecule inhibitors |
| | | of the Dishevelled- |
| | | CXXC5 interaction are |
| | | new drug candidates |
| | | for bone anabolic |
| | | osteoporosis therapy” of |
| | | Materials and Methods section |
| Library | Library size | 2280 compounds assayed |
| | | in 96-well plates |
| | | as single compounds at |
| | | 10 mM in DMSO |
| | Library composition | Small molecules |
| | Source | ChemDiv and Sigma |
| | | LOPAC 1280 |
| Screen | Format | 96-well black |
| | | polystyrene plates |
| | Concentration(s) tested | Constant 30 μM |
| | | concentration, |
| | | 0.3% DMSO |
| | Plate controls | DMSO-treated group |
| | Reagent/compound | Reagents and compounds |
| | dispensing system | were dispensed |
| | | manually |
| | Detection instrument and | FLUOstar OPTIMA |
| | software | (BMG LABTECH) |
| | Assay validation/QC | Z-factor > 0.7 |
| | Correction factors | N/A |
| | Normalization | The sample result was |
| | | normalized to |
| | | positive control and |
| | | is represented as % |
| | | CXXC5-DVL interaction |
| Post-HTS analysis | Hit criteria | <10% inhibition |
| | Hit rate | 1% |

In silico docking (Flexible docking). NMR structure of DVL PDZ domain with known ligand, sulindac, was obtained from the Protein Data Bank (PDB code: 2KAW). BIO, I3O, and KY19382 were docked to the sulindac binding site of PDZ domain by using Flexible docking method in Discovery Studio software adopting the CHARMm force field (Dassault Systemes BIOVIA, Discovery Studio Modeling Environment, Release 2017, San Diego: Dassault Systemes, 2016). For the docking analysis, active site was defined within 10.6 Å sphere centered from the ligand and the core site was redefined including residues I264, S265, I266, L321, R324, and V325, known as make up the receptor-ligand interaction. Based on the docking results, various scoring functions (Ligscore1_Dreiding, Ligscore2_Dreiding, PLP1, PLP2, PMF, DOCKSCORE) were determined to calculate binding energy the most predictive binding mode.

### Synthesis of 5,6-dichloroindirubin-3′-methoxime (KY19382)

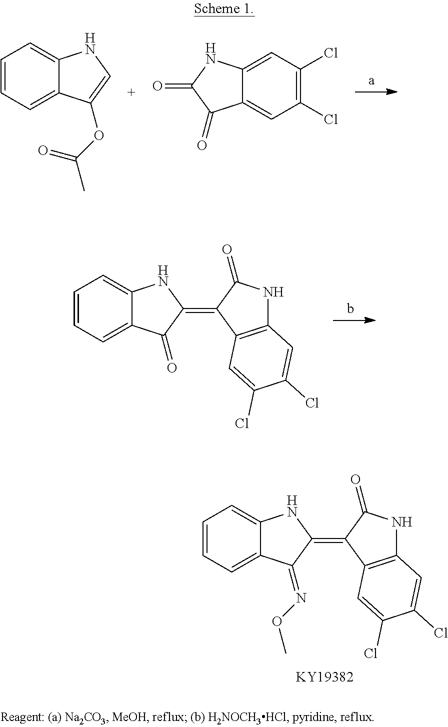 [SMILES: CC(=O)Oc1c[nH]c2ccccc12 ; O=C1Nc2cc(Cl)c(Cl)cc2C1=O ; O=C1Nc2cc(Cl)c(Cl)cc2/C1=C1/Nc2ccccc2C1=O ; CO/N=C1/C(=C2/C(=O)Nc3cc(Cl)c(Cl)cc32)Nc2ccccc21]

To a solution of 3-acetoxyindole (405 mg, 2.32 mmol) in methanol (0.025 M), 5,6-dichloroisatin (500 mg, 2.32 mmol) and sodium carbonate (613 mg, 5.79 mmol) were added. The reaction mixture was refluxed for overnight. The dark precipitate was filtered and washed with aqueous ethanol and recrystallized by ethanol and H₂O solution (1:1 (v/v)). The desired compound, 5,6-dichloro-indirubin was dried in vacuo to give as a purple solid with 63% yield. ¹H NMR (400 MHz, DMSO-d6) δ 11.10 (s, 2H), 8.89 (s, 1H), 10.70 (s, 1H), 7.63 (d, 1H, J=8.0 Hz), 7.60-7.55 (m, 1H), 7.40 (d, 1H, J=8.0 Hz), 7.05-7.01 (m, 1H). Next, to a solution of 5,6-dichloro-indirubin (200 mg, 0.61 mmol) in pyridine (0.15 M) was added the anhydrous hydroxylamine hydrochloride (509 mg, 6.1 mmol). The reaction mixture was refluxed (120° C.) for overnight. After the reaction was completed, the solvent was evaporated under reduced pressure and the residue was fully dissolved in ethyl acetate and water with sonication. The reaction mixture was extracted using ethyl acetate (100 ml×2) and washed with saturated aqueous sodium bicarbonate solution (200 ml), dried over anhydrous MgSO₄. The crude product was recrystallized with methanol and hexane solution, and the desired product, 5,6-dichloroindirubin-3′-methoxime was dried in vacuo to give as a reddish brown solid with 31% yield. ¹H NMR (400 MHz, DMSO-d6) δ 11.63 (s, 1H), 10.92 (s, 1H), 8.73 (s, 1H), 8.02 (d, 1H, J =4.0 Hz), 7.41-7.37 (m, 2H), 7.02-6.98 (m, 1H), 6.94 (s, 1H), 4.33 (s, 3H).

### Synthesis of 5-methoxylindirubin-3′-oxim (A3334)

### Synthesis of intermediate product, 5′-methoxy-[2,3′-biindolinylidene]-2′,3-dione

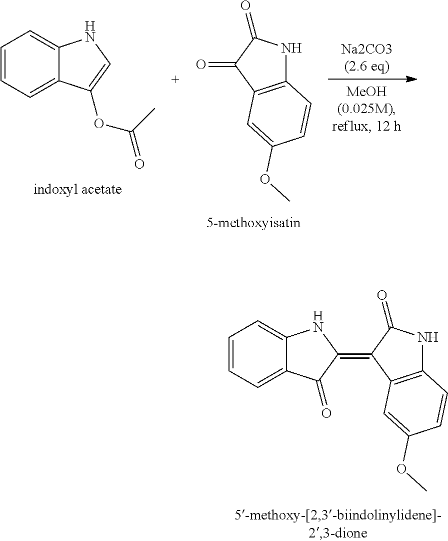 [SMILES: CC(=O)Oc1c[nH]c2ccccc12 ; COc1ccc2c(c1)C(=O)C(=O)N2 ; COc1ccc2c(c1)/C(=C1/Nc3ccccc3C1=O)C(=O)N2]

5-methoxyisatin (1000 mg, 5.65 mmol) was added to a 250-mL round bottom flask and dissolved in methanol (225 mL), followed by the addition of indoxyl acetate (989 mg, 5.65 mmol) and sodium carbonate (Na₂CO₃) (1496 mg, 14.11 mmol), and the mixture is stirred at 65° C. for 12 hours. The reaction is terminated using TLC (Rf=0.4, ethyl acetate/hexane=½ (v/v)) and the product is allowed to cool down on ice until a lump of crystals is formed. After the crystals are formed and the solvent is removed by filtration, the filtrate is discarded, and the product is washed several times with a solvent (ethanol/water=1/1 (v/v)). The product was filtered and dried in a vacuum pump and used in the next step without further purification.

### Synthesis of A3334

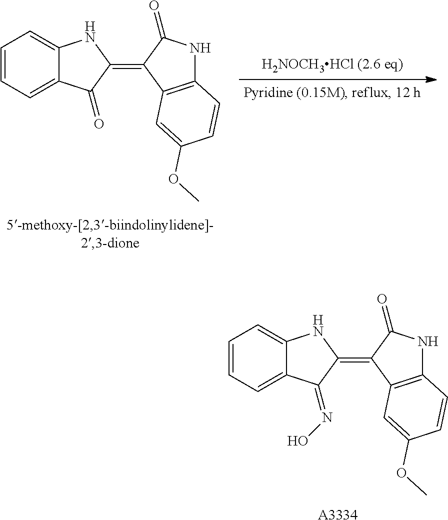 [SMILES: COc1ccc2c(c1)/C(=C1/Nc3ccccc3C1=O)C(=O)N2 ; COc1ccc2c(c1)/C(=C1/Nc3ccccc3/C1=N\O)C(=O)N2 ; "A3334"]

5′-methoxy-[2,3′-biindolinylidene]-2′,3-dione) (670 mg, 2.29 mmol) was added to a 100-mL round bottom flask and dissolved in pyridine (27 ml), followed by addition of H₂NOCH₃—HCl (3186 mg, 45.85 mmol) and the mixture was stirred at 120° C. for 12 hours. The reaction is terminated using TLC (Rf=0.5, ethyl acetate/hexane= 1/1 (v/v)) and the temperature of the reaction solution is lowered to room temperature. After evaporation of the pyridine solvent, the product was dissolved in water and ethylacetate for 30 minutes using ultrasonic waves. The product was extracted twice with ethyl acetate and washed with saturated NaHCO₃ solution. The extracted solution is dehydrated with anhydrous magnesium sulfate, the solvent is evaporated and recrystallized using methanol and nucleic acid. The product was dried in a vacuum pump and red solid A3334 can be obtained (420 mg) in 59% yield.

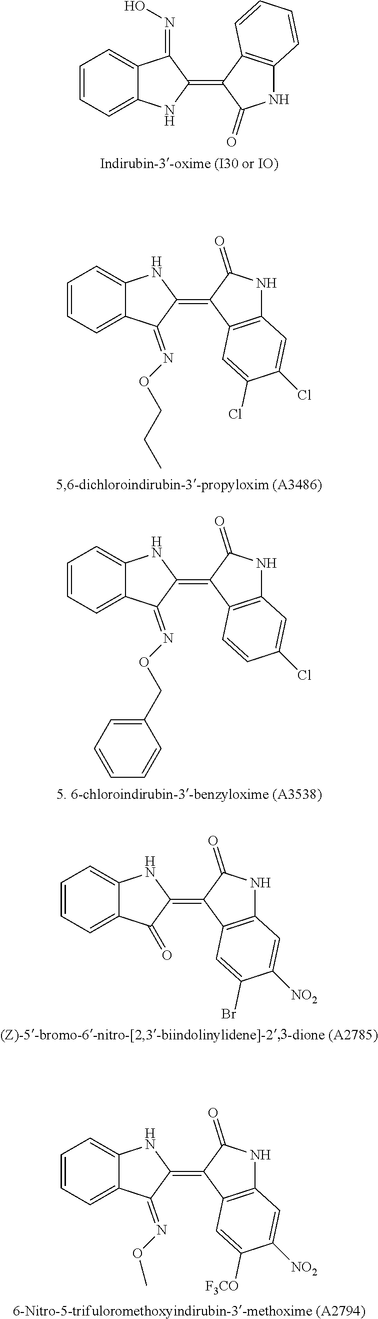 [SMILES: O=C1Nc2ccccc2/C1=C1/Nc2ccccc2/C1=N\O ; CCCO/N=C1/C(=C2/C(=O)Nc3cc(Cl)c(Cl)cc32)Nc2ccccc21 ; O=C1Nc2cc(Cl)ccc2/C1=C1/Nc2ccccc2/C1=N\OCc1ccccc1 ; O=C1Nc2cc([N+](=O)[O-])c(Br)cc2/C1=C1/Nc2ccccc2C1=O ; CO/N=C1/C(=C2/C(=O)Nc3cc([N+](=O)[O-])c(OC(F)(F)F)cc32)Nc2ccccc21]

In vivo pharmacokinetics of KY19382. Animal studies were approved by the Institutional Animal Care and use committee at KRIBB. Briefly, specific pathogen-free male Sprague-Dawley (SD) rats (10 weeks old, body weight 298-315 g), purchased from Koatech Co. (Kyeonggi, Republic of Korea), were given a single dose of KY19382 intravenously (iv, 1 mg/kg, n=3) or intraperitoneal (ip, 5 mg/kg, n=3). Dosing solutions were prepared in dimethylacetamide (DMAC)/polyethylene glycol 400 (PEG400) (20/80, v/v %) for administrations, and administered at dosing volumes of 5 and 10 mL/kg for iv and ip, respectively. A 100 μl aliquot of each plasma sample was prepared, and three volumes of ice-cold acetonitrile containing carbamazepine (internal standard) were added. The mixture was centrifuged at 910 g for 10 min, and the supernatant was subjected to LC-MS/MS analysis. Pharmacokinetic parameters were calculated by standard noncompartmental analysis of plasma concentration-time profiles using Kinetica 4.4.1 (Thermo Fisher Scientific, Inc., Woburn, Mass., USA). The areas under the plasma concentration-time curves (AUC) were calculated by the linear-trapezoidal method. Systemic plasma clearance (CLP) was calculated as follows: CLP=dose/AUCinf. Terminal elimination half-life (t½) was calculated by the following equation: t½=0.693/λZ where λZ is the terminal disposition rate constant. Volume of distribution at steady state (VSS) was calculated as follows: VSS=dose×AUMCinf/(AUCinf)2, where AUMCinf is the area under the first moment of the plasma concentration-time curve extrapolated to infinity Bioavailability (F) was calculated as follows: F (%)=(AUCip/AUCiv)(doseiv/doseip)×100.

The hallmark of growth plate senescence includes a decline in the overall height of the growth plate with a decrease in the number of resting, proliferative, and hypertrophic chondrocytes per column and an increase in the spacing between adjacent chondrocyte columns. Unlike “senescence,” which generally refers to specific cellular program, the term “growth plate senescence” indicates a physiological loss of function that occurs with increasing age. See e.g., Gafni R I, et al. (2001) Catch-up growth is associated with delayed senescence of the growth plate in rabbits. PEDIATR RES 50: 618-623 and Nilsson, et al. (2004) Fundamental limits on longitudinal bone growth: growth plate senescence and epiphyseal fusion. TRENDS ENDOCRINOL METAB 15: 370-374. Although many children undergo early growth plate senescence and reach a short height in adulthood due to precocious puberty, the mechanism of these phenomena is poorly understood. Recent studies suggest that growth plate activity is primarily regulated by paracrine factors that directly exert their function on chondrocytes within the growth plate. Wnt/β-catenin signaling has been implicated in these functions; however, the molecular mechanisms and factors controlling growth plate senescence are still unexplored.

In the instant disclosure, it was found that a negative feedback regulator of the Wnt/β-catenin pathway, CXXC5, gradually increased with suppression of β-catenin in growth plate chondrocytes at the later stages of puberty. Moreover, upregulation of CXXC5 was in part mediated by estrogen, a sex hormone that can be increased with pubertal progression.

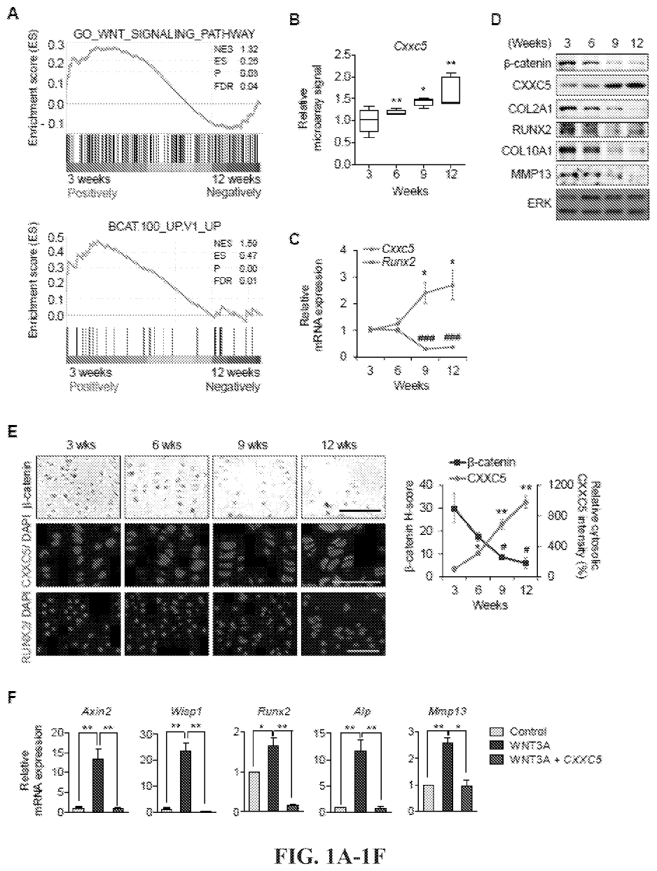

CXXC5 expression progressively increases in the growth plate at later stages of puberty. To elucidate the involvement of Wnt/β-catenin signaling in growth plate senescence, gene set enrichment analysis was used and the expression profiles of Wnt-responsive genes in the growth plates of 3-week-old (pre- and early puberty) and 12-week-old (early adulthood) rats (GEO: GSE16981) was investigated. Referring now to FIG. 1A, the signatures of Wnt/β-catenin signaling-activated genes were significantly downregulated in the growth plates of the 12-week-old rats. Moreover, the mRNA level of Cxxc5, a negative regulator of Wnt/β-catenin signaling, was gradually elevated during pubertal progression (GEO: GSE16981) (FIG. 1B), showing a statistically significant increase at 12 weeks compared to other inhibitors of Wnt/β-catenin signaling (Apcdd1, Cxxc4, Dkk2, Igfbp4, Sfrp family, Shisa family, Sost, and Wif1) (Table 4).

TABLE 4
Analysis of mRNA expression
levels of Wnt inhibitors

| | Upregulated gene |
|---|---|
| | in 12 weeks |
| Wnt inhibitors | (P < 0.05) |
| Apcdd1 | — |
| Cxxc4 | — |
| Cxxc5 | P = 6.45E−04 |
| Dkk2 | — |
| Igfbp4 | — |
| Sfrp1 | — |
| Sfrp2 | — |
| Sfrp4 | — |
| Sfrp5 | P = 5.74E−03 |
| Shisa3 | — |
| Shisa4 | — |
| Shisa5 | — |
| Shisa7 | — |
| Shisa8 | — |
| Shisa9 | — |
| Sost | — |
| Wifl | P = 6.58E−03 |

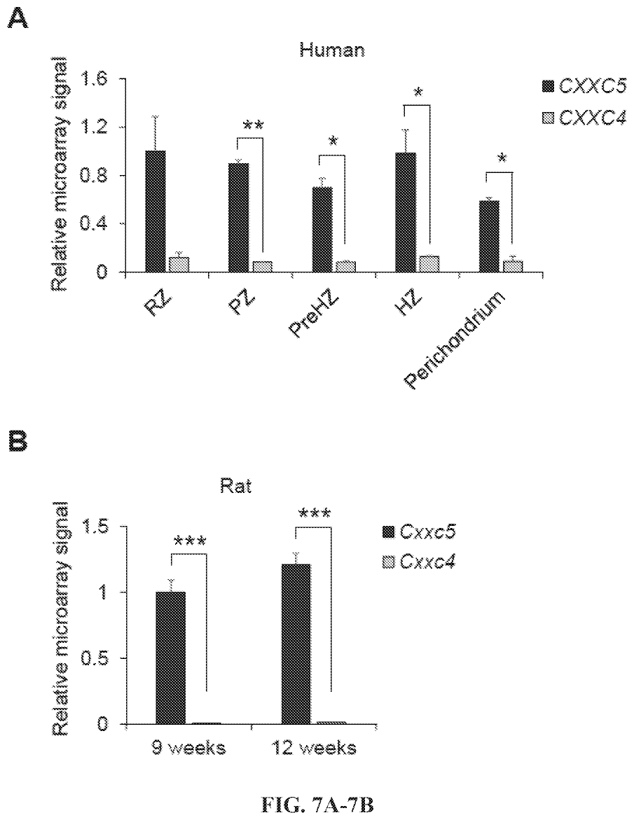

CXXC4, a structural and functional analog of CXXC5 that also functions as a negative regulator of Wnt/β-catenin signaling, was not significantly induced at puberty in the growth plate zones of humans or rats when compared with CXXC5 (FIGS. 7A and 7B).

To examine the pubertal period in more detail, the growth plates of proximal tibiae from 3-, 6-, 9-, and 12-week-old mice were collected and subjected to additional analyses. Referring now to FIG. 1C, upregulation of Cxxc5 and downregulation of Runx2 (Wnt/β-catenin signaling-targeting chondrogenic differentiation marker) were confirmed by qRT-PCR analyses at the later stages of pubertal progression compared to 3-week-old mice. Immunoblot analyses also showed that CXXC5 gradually increased with the decrement of β-catenin and chondrogenic markers including COL2A1., RUNX2, COL10A1, and MMP13 in the growth plates of mice undergoing pubertal progression (FIG. 1D). The inverse correlation between CXXC5 and Wnt/β-catenin signaling was verified by immunohistochemical analyses showing progressive increase of cytosolic CXXC5 with the gradual decrease of nuclear β-catenin and its target RIJNX2 in the growth plates of 3- to 12-week-old mice (FIG. 1E). Next, the inhibitory effects of CXXC5 on Wnt/β-catenin pathway and chondrogenic differentiation in cell level were examined. Referring now to FIG. 1F, the WNT3A-inducd increase in Wnt/β-catenin signaling target genes, Axin2 and Wisp1, were suppressed by CXXC5 overexpression. Further, the WNT3A-induced transcriptional increase in chondrogenic differentiation markers, such as Runx2, Alp, and Mmp13, were also suppressed by CXXC5 overexpression.

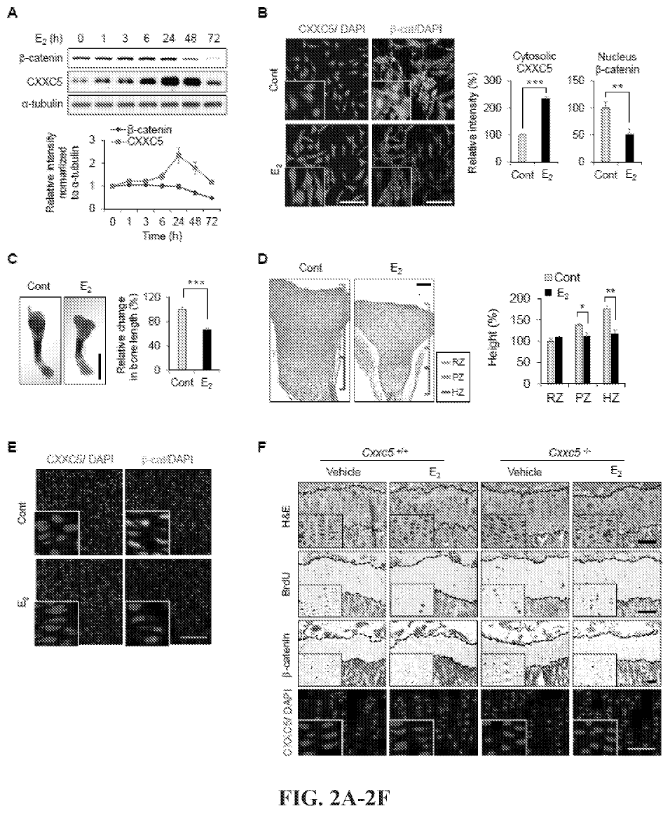

CXXC5 mediates growth plate senescence induced by the sexual hormone, estrogen. Estrogen, a hormone involved in sexual maturation, is known to be elevated at puberty and can play a role in growth plate senescence. The effect of 17β-estradiol (E₂), a major estrogenic hormone in the circulation, were examined on CXXC5 expression in the human chondrocyte cell line, C28/I2. Referring now to FIG. 2A, treatment of E₂ induced expression of CXXC5 in a time-dependent manner, achieving a maximal level at 24 hours. Further, β-catenin level was reduced following 24 hours of E₂ treatment. As shown in FIG. 2B, E₂ prominently elevated cytosolic CXXC5 and repressed cytosolic and nuclear β-catenin. The role of E₂ on growth plate senescence was further confirmed by use of an ex-vivo tibial culture system that demonstrated reduced tibial length with decreased height of proliferative and hypertrophic zones in the growth plate following E₂ treatment (FIGS. 2C and D). The induction of cytosolic CXXC5 and the decrement of nuclear β-catenin in the chondrocytes of E₂-treated growth plates supports the previously identified relationship between growth plate senescence and inactivation of Wnt/β-catenin signaling (compare FIG. 2E with FIG. 1E).

To verify involvement of estrogen in CXXC5 expression and growth plate senescence, the effects of E₂ treatment were compared in 6-week-old Cxxc5^{+/+} and Cxxc5^{-/-} mice. Referring now to FIG. 2F, E₂-induced structural senescence of the tibial growth plate was shown in Cxxc5^{+/+} mice but was hardly observed in Cxxc5^{-/-} mice. In addition, there were no significant changes in BrdU incorporation and β-catenin expression in E2-treated. Cxxc5^{-/-} mice. These results show that CXXC5 mediates growth plate senescence upon induction by estrogen.

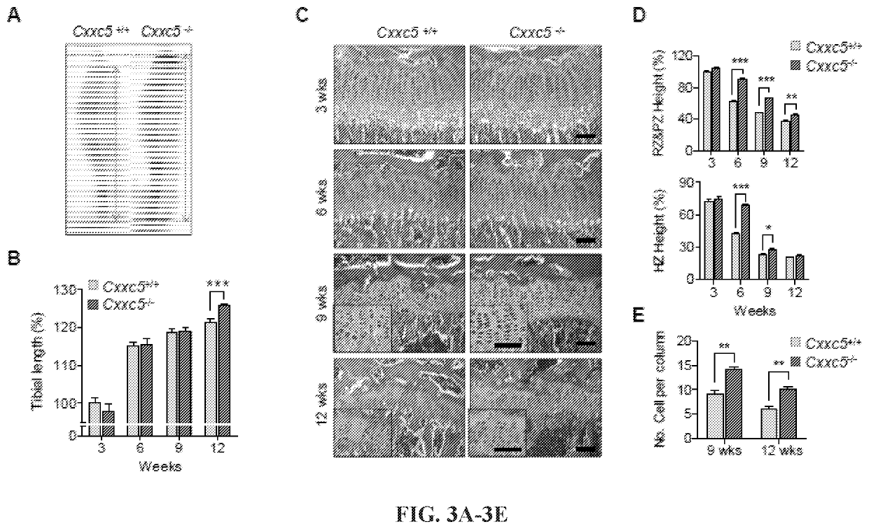

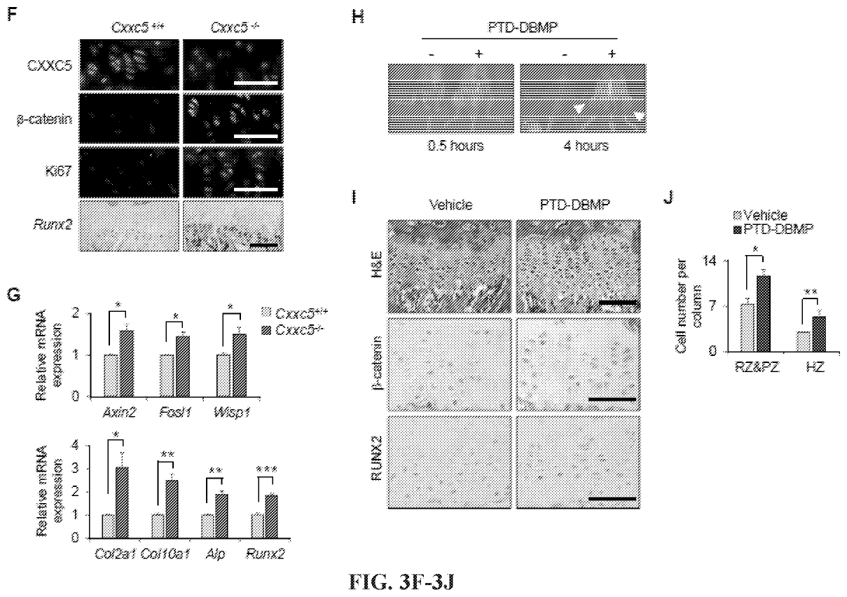

CXXC5 plays a key role in suppression of longitudinal bone growth at late puberty. To further define the role of CXXC5 in growth plate senescence during pubertal progression, longitudinal bone growth and growth plate senescence in Cxxc5^{+/+} and Cxxc5^{-/-} mice were assessed. Referring now to FIG. 3, Cxxc5^{-/-} mice showed significantly enhanced tibial lengths at 12 weeks of age (FIGS. 3A and 3B). With aging, growth plates of Cxxc5^{+/+} mice naturally underwent structural senescence as monitored by gradual reduction of the height of resting, proliferative, and hypertrophic zones with a concomitant decline in the number of chondrocytes in each zone (FIG. 3C-3E). However, these age-related changes were significantly delayed in Cxxc5^{-/-} mice, although the growth plates of Cxxc5^{-/-} mice did eventually undergo structural senescence with aging (FIG. 3C-3E). Referring now to FIG. 3F, the retardation of growth plate senescence by Cxxc5 deletion was further supported by marked increases of Ki67 and β-catenin protein levels together with Runx2 mRNA level in chondrocytes of the growth plates of 11-week-old Cxxc5^{-/-} mice compared to 11-week-old Cxxc5^{+/+} mice. The activation of Wnt/β-catenin signaling and the promotion of chondrogenic differentiation was further confirmed by the upregulation of Wnt/β-catenin target genes (Axin2, Fosl1, and Wisp1) and chondrogenic markers (Col2a1, Col10a1, Alp, and Runx2) in the growth plates of 9-week-old Cxxc5^{-/-} mice compared to 9-week-old Cxxc5^{-/-} mice (FIG. 3G).

CXXC5 can function as a negative regulator of Wnt/β-catenin pathway by binding to DVL. A protein transduction domain fused DVL binding motif peptide (PTD-DBMP), which interferes with the CXXC5-DVL interaction (Kim et al., 2015), were tested whether it would exert effects similar to the loss of Cxxc5 on growth plate senescence. Referring now to FIG. 3H, injection of PTD-DBMP into the growth plates of 7-week-old mice (late puberty) increased the number of resting, proliferative and hypertrophic chondrocytes per column. The injection of PTD-DBMP further increased β-catenin and RUNX2 levels in chondrocytes of the growth plate (FIGS. 3I and 3J). Overall, these results indicate that CXXC5 l plays a role in the structural senescence of the growth plate, which can be acquired by inhibition of the CXXC5-DVL interaction.

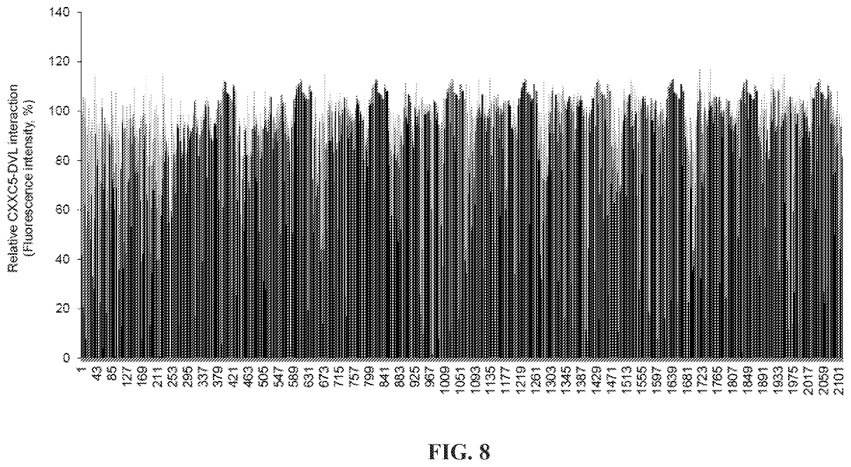

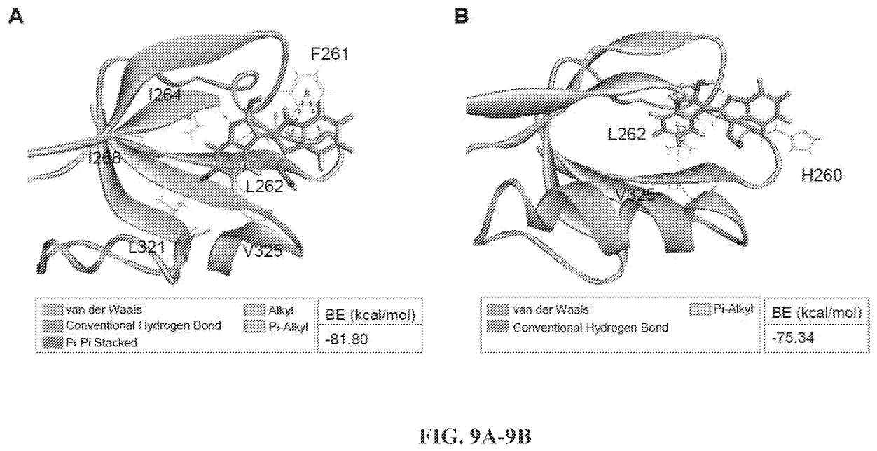

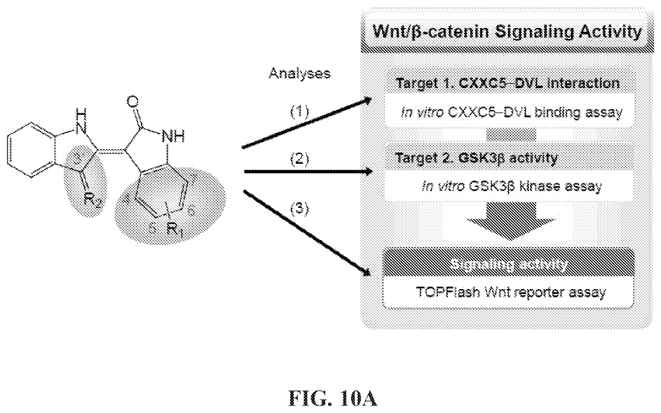

KY19382 activates Wnt/β-catenin signaling through inhibitory effects on both CXXC5-DVL interaction and GSK3β activity. To identify small molecules that mimic the function of the PTD-DBMP and delay growth plate senescence, 2,280 compounds were screened from chemical libraries (1,000 from ChemDiv and 1,280 from SigmaLOPAC) with an in vitro assay system that monitors the CXXC5-DVL interaction (Kim et al, 2016) (FIG. 8). In this screening system, the indirubin analogs 6-bromoindirubine-3′-oxime (hereinafter “BIO”) (compound 8) and indirubin-3′-oxime (hereinafter “I3O”) (compound 12), which are known GSK3β inhibitors, were identified as top-ranked positive initial hits (see also Tables 3 and 5). See, e.g., Meijer L, et al. (2003) GSK-3-selective inhibitors derived from Tyrian purple indirubins. CHEM BIOL 10: 1255-1266. As shown in FIG. 9, BIO and I3O interacted with DVL PDZ domain (Protein Data Bank [PDB]: 2KAW) in an in .silica docking modeling.

TABLE 5
List of top-ranked compounds screened through an in vitro binding assay of
chemical libraries that includes 2,280 small molecules

| | | | CXXC5-DVL |
|---|---|---|---|
| | | Empirical | interaction |
| Compound | Structure | Formula | (%) |
| 1 | 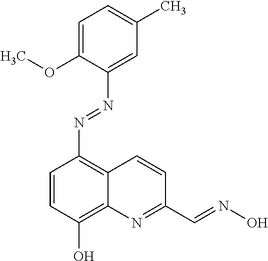 [SMILES: COc1ccc(C)cc1/N=N/c1ccc(O)c2nc(/C=N/O)ccc12] | C₁₈H₁₆N₄O₃ | 7.62 |
| 2 | 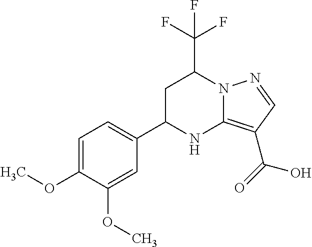 [SMILES: COc1ccc(C2CC(C(F)(F)F)n3ncc(C(=O)O)c3N2)cc1OC] | C₁₆H₁₆F₃N₃O₄ | 1.01 |
| 3 | 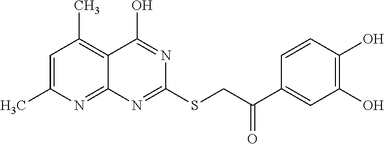 [SMILES: Cc1cc(C)c2c(O)nc(SCC(=O)c3ccc(O)c(O)c3)nc2n1] | C₁₇H₁₅N₃O₄S | 4.01 |
| 4 | 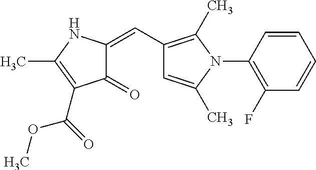 [SMILES: COC(=O)C1=C(C)N/C(=C/c2cc(C)n(-c3ccccc3F)c2C)C1=O] | C₂₀H₁₉FN₂O₃ | 4.76 |
| 5 | 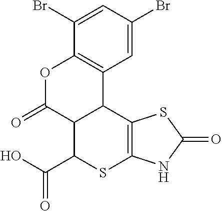 [SMILES: O=C(O)C1Sc2[nH]c(=O)sc2C2c3cc(Br)cc(Br)c3OC(=O)C12] | C₁₄H₇Br₂NO₅S₂ | 1.19 |
| 6 | 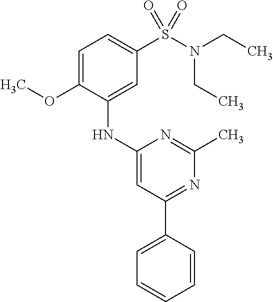 [SMILES: CCN(CC)S(=O)(=O)c1ccc(OC)c(Nc2cc(-c3ccccc3)nc(C)n2)c1] | C₂₂H₂₆N₄O₃S | 7.63 |
| 7 | 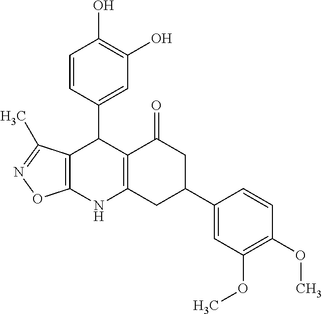 [SMILES: COc1ccc(C2CC(=O)C3=C(C2)Nc2onc(C)c2C3c2ccc(O)c(O)c2)cc1OC] | C₂₅H₂₄N₂O₆ | 0.72 |
| 8 | 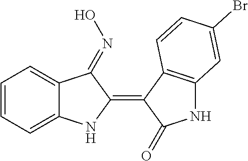 [SMILES: O=C1Nc2cc(Br)ccc2/C1=C1/Nc2ccccc2/C1=N\O] | C₁₆H₁₀BrN₃O₂ | 0 |
| 9 | 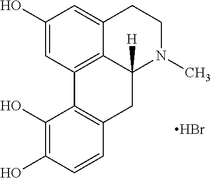 | C₁₇H₁₇NO₃•HBr | 2.5 |
| 10 | 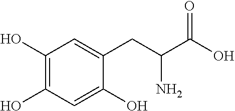 [SMILES: NC(Cc1cc(O)c(O)cc1O)C(=O)O] | C₉H₁₁NO₅ | 1.39 |
| 11 | 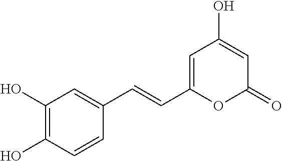 [SMILES: O=c1cc(O)cc(/C=C/c2ccc(O)c(O)c2)o1] | C₁₃H₁₀O₅ | 1.73 |
| 12 | 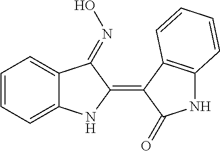 [SMILES: O=C1Nc2ccccc2/C1=C1/Nc2ccccc2/C1=N\O] | C₁₆H₁₁N₃O₂ | 7.65 |
| 13 | 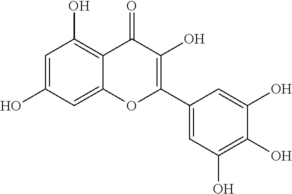 [SMILES: O=c1c(O)c(-c2cc(O)c(O)c(O)c2)oc2cc(O)cc(O)c12] | C₁₅H₁₀O₈ | 7.66 |
| 14 | 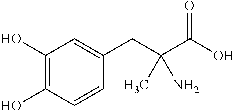 | C₁₀H₁₃NO₄ | 7.65 |
| 15 | 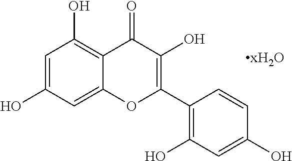 [SMILES: O=c1c(O)c(-c2ccc(O)cc2O)oc2cc(O)cc(O)c12] | C₁₅H₁₀O₇•xH₂O | 7.43 |
| 16 | 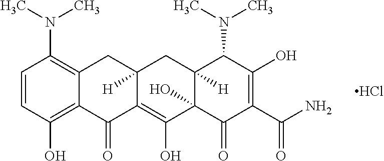 | C₂₃H₂₇N₃O₇•HCl | 4.37 |
| 17 | 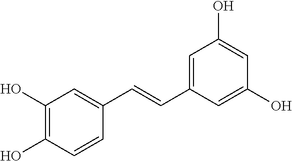 [SMILES: Oc1cc(O)cc(/C=C/c2ccc(O)c(O)c2)c1] | C₁₄H₁₂O₄ | 7.61 |
| 18 | 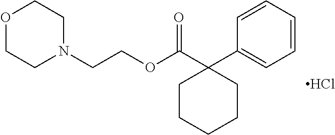 [SMILES: O=C(OCCN1CCOCC1)C1(c2ccccc2)CCCCC1 ; Cl] | C₁₉H₂₇NO₃•HC1 | 5.7 |
| 19 | 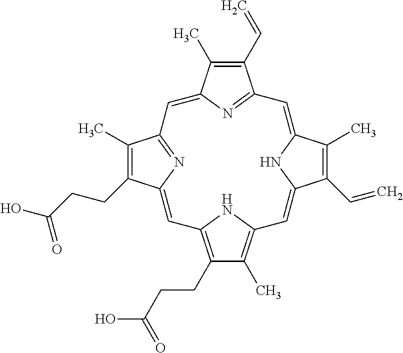 [SMILES: C=CC1=C(C)c2cc3nc(cc4[nH]c(cc5[nH]c(cc1n2)c(C)c5C=C)c(C)c4CCC(=O)O)C(CCC(=O)O)=C3C] | C₃₄H₃₄N₄O₄ | 2.47 |

TABLE 6
List of chemically synthesized compounds shown to at least partially inhibit the activity of CXXC5-DVL.

| | Compound | | R1 | | | | R2 |
|---|---|---|---|---|---|---|---|
| | # | IUPAC name | 4 | 5 | 6 | 7 | 3′ |
| C | Indirubin | Indirubin | H | H | H | H | O |
| 1 | A2735 | 6-Chloro-5-nitroindirubin | H | NO₂ | Cl | H | O |
| 2 | A2736 | 6-Chloro-5-nitroindirubin-3′-oxime | H | NO₂ | Cl | H | NOH |
| 3 | A2941 | 5,6-dichloroindirubin | H | Cl | Cl | H | O |
| 4 | A3050 | 5,6-dichloroindirubin-3′-oxime | H | Cl | Cl | H | NOH |
| 5 | KY19382 | 5,6-dichloroindirubin-3′-methoxime | H | Cl | Cl | H | NOCH₃ |
| 6 | A3471 | 5,6-dichloroindirubin-3′-ethyloxime | H | Cl | Cl | H | NOCH₂CH₃ |
| 7 | A3486 | 5,6-dichloroindirubin-3′-propyloxime | H | Cl | Cl | H | NOCH₂CH₂CH₃ |
| 8 | A2813 | 6-Chloroindirubin | H | H | Cl | H | O |
| 9 | A2853 | 6-Chloroindirubin-3′-oxime | H | H | Cl | H | NOH |
| 10 | A2793 | 6-Chloroindirubin-3′-methoxime | H | H | Cl | H | NOCH₃ |
| 11 | A3473 | 6-Chloroindirubin-3′-ethyloxime | H | H | Cl | H | NOCH₂CH₃ |
| 12 | A3481 | 6-Chloroindirubin-3′-propyloxime | H | H | Cl | H | NOCH₂CH₂CH₃ |
| 13 | A3538 | 6-Chloroidirubin-3′-benzyloxime | H | H | Cl | H | NOCH₂Ph |
| 14 | A2851 | 5-Chloroindirubin | H | Cl | H | H | O |
| 15 | A3439 | 5-Chloroindirubin-3′-oxime | H | Cl | H | H | NOH |
| 16 | A3440 | 5-Chloroindirubin-3′-methoxime | H | Cl | H | H | NOCH₃ |
| 17 | A3470 | 5-Chloroindirubin-3′-ethyloxime | H | Cl | H | H | NOCH₂CH₃ |
| 18 | A3485 | 5-Chloroindirubin-3′-propyloxime | H | Cl | H | H | NOCH₂CH₂CH₃ |
| 19 | A3536 | 5-Chloroindirubin-3′-benzyloxime | H | Cl | H | H | NOCH₂Ph |
| 20 | A3331 | 5-Methoxyindirubin | H | OCH₃ | H | H | O |
| 21 | A3334 | 5-Methoxyindirubin-3′-oxime | H | OCH₃ | H | H | NOH |
| 22 | A3441 | 5-Methoxyindirubin-3′-methoxime | H | OCH₃ | H | H | NOCH₃ |
| 23 | A3484 | 5-Methoxyindirubin-3′-ethyloxime | H | OCH₃ | H | H | NOCH₂CH₃ |
| 24 | A3483 | 5-Methoxyindirubin-3′-proyloxime | H | OCH₃ | H | H | NOCH₂CH₂CH₃ |
| 25 | A3330 | 5-Methylindirubin | H | CH₃ | H | H | O |
| 26 | A3335 | 5-Methylindirubin-3′-oxime | H | CH₃ | H | H | NOH |
| 27 | A3442 | 5-Methylindirubin-3′-methoxime | H | CH₃ | H | H | NOCH₃ |
| 28 | A3533 | 5-Methylindirubin-3′-ethyloxime | H | CH₃ | H | H | NOCH₂CH₃ |
| 29 | A3534 | 5-Methylindirubin-3′-propyloxime | H | CH₃ | H | H | NOCH₂CH₂CH₃ |
| 30 | A3535 | 5-Methylindirubin-3′-benzyloxime | H | CH₃ | H | H | NOCH₂Ph |

C: control

TABLE 7
List of chemically synthesized compounds shown to at least partially inhibit the activity of CXXC5-DVL.

| | Compound | | R1 | | | | R2 |
|---|---|---|---|---|---|---|---|
| | # | IUPAC name | 4 | 5 | 6 | 7 | 3′ |
| C | I3O | Indirubin-3′-oxime | H | H | H | H | NOH |
| 31 | A3332 | 5-Bromoindirubin | H | Br | H | H | O |
| 32 | A3390 | 5-bromoindirubin-3′-oxime | H | Br | H | H | NOH |
| 33 | A3391 | 5-bromoindirubin-3′-methoxime | H | Br | H | H | NOCH₃ |
| 34 | A3472 | 5-bromoindirubin-3′-ethyloxime | H | Br | H | H | NOCH₂CH₃ |
| 35 | A3482 | 5-bromoindirubin-3′-propyloxime | H | Br | H | H | NOCH₂CH₂CH₃ |
| 36 | A3537 | 5-bromoindirubin-3′-benzyloxime | H | Br | H | H | NOCH₂Ph |
| 37 | A2784 | 5-Chloro-6-nitroindirubin | H | Cl | NO₂ | H | O |
| 38 | A2848 | 5-Chloro-6-nitroindirubin-3′-oxime | H | Cl | NO₂ | H | NOH |
| 39 | A3049 | 5-Chloro-6-nitroindirubin-3′-methoxime | H | Cl | NO₂ | H | NOCH₃ |
| 40 | A2849 | 5-Nitroindirubin | H | H | NO₂ | H | O |
| 41 | A2854 | 5-Nitroindirubin-3′-oxime | H | H | NO₂ | H | NOH |
| 42 | A3333 | 5-Trifluoromethoxyindirubin | H | OCF₃ | H | H | O |
| 43 | A3392 | 5-Trifluoromethoxyindirubin | H | OCF₃ | H | H | NOH |
| 44 | A3393 | 5-Trifluoromethoxyindirubin-3′-methoxime | H | OCF₃ | H | H | NOCH₃ |
| 45 | A2942 | 6-Methylindirubin | H | H | CH₃ | H | O |
| 46 | A2943 | 6-Methylindirubin-3′-oxime | H | H | CH₃ | H | NOH |
| 47 | A2944 | 6-Methylindirubin-3′-methoxime | H | H | CH₃ | H | NOCH₃ |
| 48 | A2852 | 6-Methyl-5-nitroindirubin | H | NO₂ | CH₃ | H | O |
| 49 | A3336 | 6-Methyl-5-nitroindirubin-3′-oxime | H | NO₂ | CH₃ | H | NOH |
| 50 | A3337 | 6-Methyl-5-nitroindirubin-3′-methoxime | H | NO₂ | CH₃ | H | NOCH₃ |
| 51 | A2802 | 6-Nitro-5-Trifluoromethoxyindirubin | H | OCF₃ | NO₂ | H | O |
| 52 | A2801 | 6-Nitro-5-trifluoromethoxyindirubin-3′-oxime | H | OCF₃ | NO₂ | H | NOH |
| 53 | A2794 | 6-Nitro-5-trifluoromethoxyindirubin-3′-methoxime | H | OCF₃ | NO₂ | H | NOCH₃ |
| 54 | A3307 | Indirubin-7-carboxylic acid | H | H | H | COOH | O |
| 55 | A3309 | Indirubin-7-carboxylic acid-3′-oxime | H | H | H | COOH | NOH |
| 56 | A3308 | 7-Trifluoromethylindirubin | H | H | H | CF₃ | O |
| 57 | A3310 | 7-Trifluoromethylindirubin-3′-oxime | H | H | H | CF₃ | NOH |
| 58 | A3311 | 4-Bromoindirubin | Br | H | H | H | O |
| 59 | A2783 | 4-Bromoindirubin-3′-oxime | Br | H | H | H | NOH |
| 60 | A3312 | 4-Chloroindirubin | H | H | H | H | O |

C: control

TABLE 8
List of chemically synthesized compounds
shown to at least partially inhibit the
activity
of CXXC5-DVL

| No. | Compound # | IUPAC Name | 3′ moiety |
|---|---|---|---|
| 1 | A4664 | 5-Fluoroindirubin | O |
| 2 | A4665 | 5-Fluoroindirubin-3′-oxime | NOH |
| 3 | A4666 | 6-Bromoindirubin | O |
| 4 | A4667 | 6-Bromoindirubin-3′-oxime | NOH |

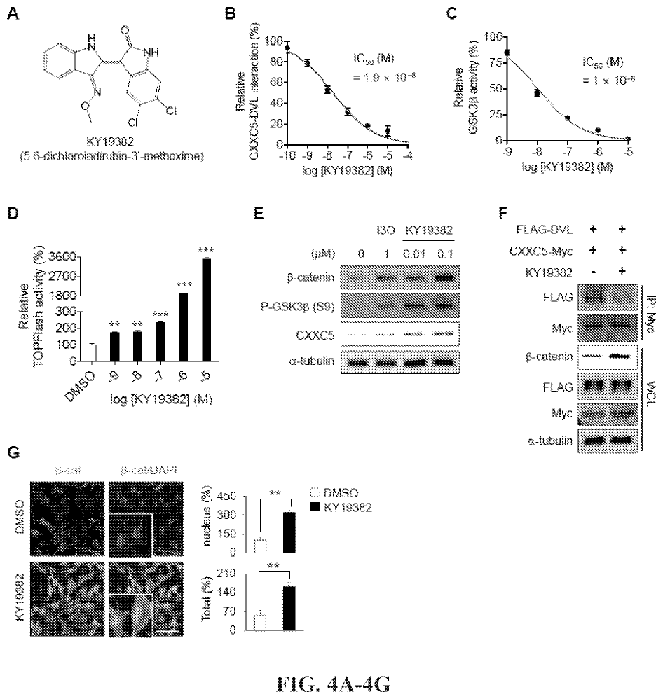

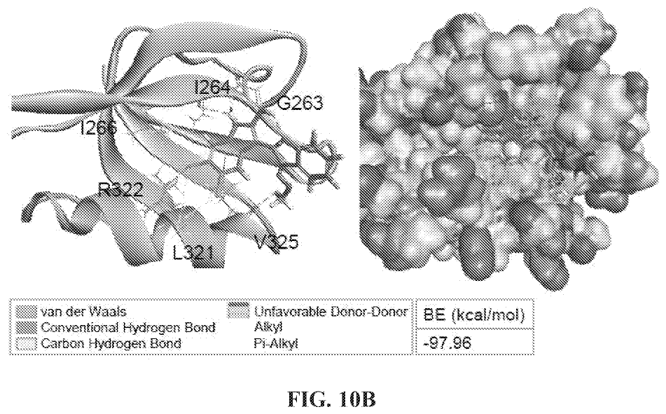

To obtain functionally improved compound, about 60 indirubin derivatives were newly synthesized by replacing the functional groups at the R₁ and R₂ sites of the indirubin backbone based on the structure of BIO and I3O (FIG. 10A and FIG. 14). By evaluating them for in vitro CXXC5-DVL binding activity, in vitro GSK3β kinase activity, and TOPFlash Wnt reporter activity, 5, 6-dichloroindirubin-3′-methoxime (KY19382; FIG. 4A) were obtained as an optimal compound for further investigation. Referring now to FIG. 4, KY19382 markedly inhibited both in vitro CXXC5-DVL interaction (IC₅₀ of KY19382=1.9×10⁻⁸M; FIG. 4B) and in vitro GSK3β activity (IC₅₀ of KY19382=1×10⁻⁸ M; FIG. 4C) with the strong enhancement of the TOPFlash Wnt reporter activity (FIG. 4D).

Possible binding sites for KY19382 on the DVL PDZ, domain (Protein Data Bank [PDB]: 2KAW) were further characterized using the in silky) docking program (FIG. 10B). Structural simulations of the KY19382-DVL PDZ, complex revealed that residues involved in the interaction with KY19382 were similar to the DBMP-binding sites (Kim et al., 2016). Compared to BIO or I3O, the estimated binding energy for the KY19382-DVL PDZ, complex was improved (BIO=−81.80 kcal mol⁻¹ or I3O=−75.34 kcal mol⁻¹ vs KY19382=−97.96 kcal mol⁻¹) (compare FIG. 9 with FIG. 10B).

Referring now to FIG. 4, The role of KY19382 in Wnt/β-catenin signaling pathway was further verified by the increment of β-catenin with the inactivation of GSK3β (FIG. 4E) and the interruption of the CXXC5-DVL interaction (FIG. 4F), resulting in the elevated nuclear translocation of β-catenin in ATDCS cells (FIG. 4G). While not wishing to be bound by any theory, the activation of β-catenin pathway by KY19382 treatment is likely dependent on both the inactivation of GSK3β and the interruption of the CXXC5-DVL interaction.

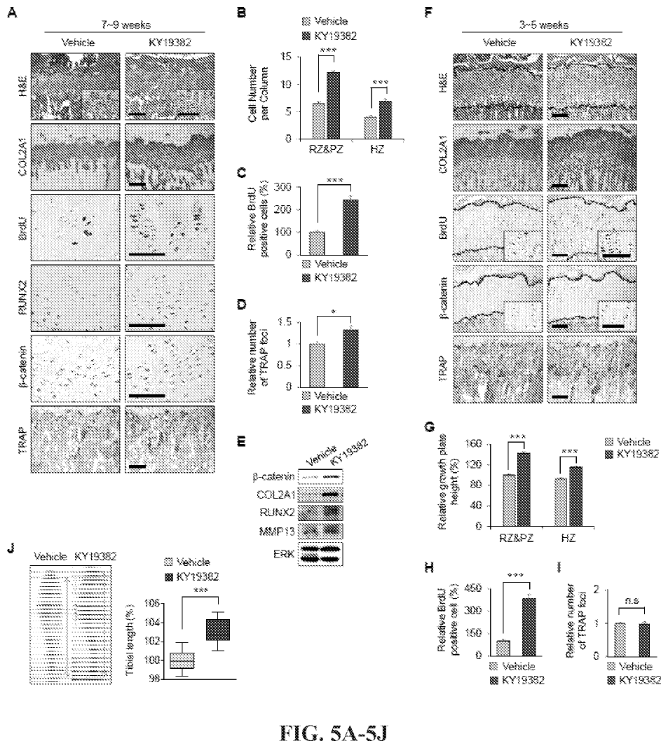

KY19382 delays growth plate senescence and promotes longitudinal bone growth. To investigate the effects of KY19382 on growth plate senescence, 0.1 mg/kg KY19382 was intraperitoneally injected into the growth plates of 7-week-old mice (late puberty) daily for 2 weeks. Referring now to FIG. 5, the total growth plate height, monitored by COL2A1 immunostaining, was significantly increased by KY19382 treatment (FIG. 5A). This effect was confirmed by increased numbers of both proliferative and hypertrophic chondrocytes per column, assessed by BrdU- and RUNX2-positive cells, respectively (FIG. 5A-5C). Along with these effects, nuclear β-catenin was dramatically increased in the growth plate chondrocytes by KY19382 treatment (FIG. 5A). Immunoblot analyses also showed that KY19382 increased β-catenin and chondrogenic markers, such as COL2A1, RUNX2, and MMP13, in the growth plate (FIG. 5E). These functional and structural changes demonstrate the ability of KY19382 in delaying growth plate senescence.

Next, the effects of KY19382 were tested in rapidly growing young mice by administering 0.1 mg/kg KY19382 at 3 weeks of age (early puberty) daily for 2 weeks. With the increase of total growth plate height, as evidenced by COL2A1 expression, the height of each growth plate zone and BrdU-positive cells were elevated in KY19382-treated mice (FIG. 5F-H). As observed in older mice, β-catenin-expressing chondrocytes were also increased by KY19382 treatment (FIG. 5F).

To exclude any possibility that the expanded HZ (hypertrophic zone) was a result of delayed cartilage resorption, TRAP staining in tibiae sections were performed. Referring now to FIG. 5I, the number of TRAP-positive foci in the growth plate/trabecular interface was not different between the groups, indicating that KY19382 did not affect the cartilage resorption of rapidly growing young mice. However, older mice treated with KY19382 from 7 weeks of age to 9 weeks of age exhibited elevated TRAP-positive foci compared to vehicle-treated mice (FIGS. 5A and 5D). These effects showed that the overall process of growth plate maturation, including preparation of the space to he replaced by osteoblastic bone formation, was activated by KY19382 treatment in spite of the senescent growth plate of late pubertal mice.

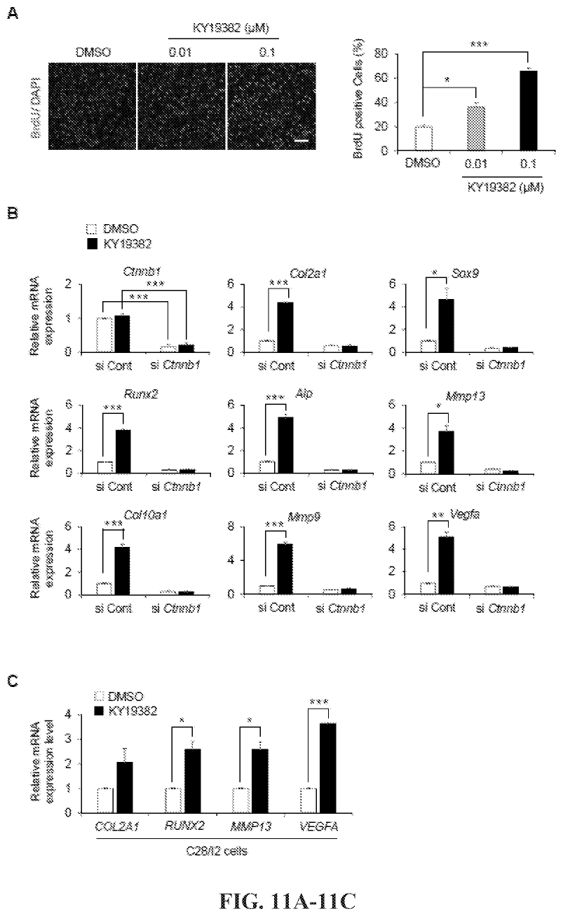

Referring now to FIG. 11, the role of KY19382 on chondrocyte proliferation was further verified in vitro by the enhanced number of BrdU-positive ATDC5 cells following KY19382 treatment (FIG. 11A). In addition, the mRNA levels of chondrogenic markers were upregulated by KY19382 in ATDC5 and C28/I2 cells (FIGS. 11B and 11C). As shown in FIG. 11B, these effects were abolished by siRNA-mediated Ctnnb1 knock-down.

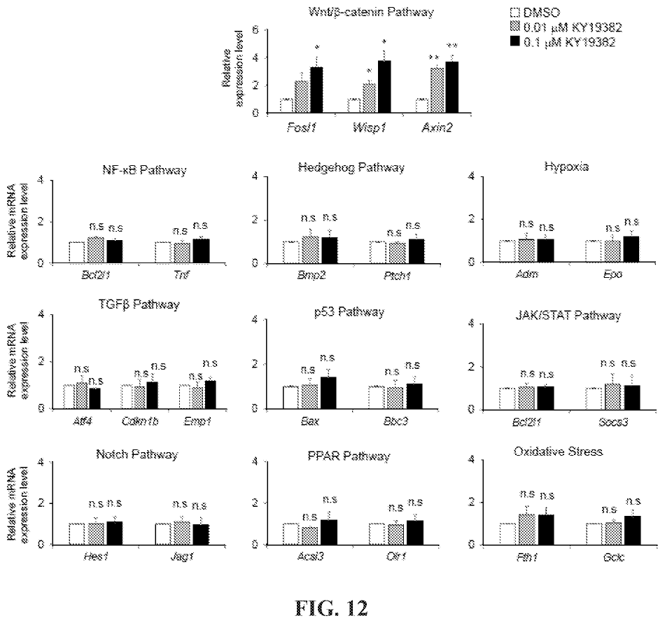

Referring now to FIG. 12, off-target effects of KY19382 were also explored by measuring mRNA levels of target genes for various signaling pathways in KY19382-treated ATDC5 cells. Although KY19382 markedly increased expression levels of Wnt/β-catenin target genes, such as Fosl1, Wisp1, and Axin2, the 19 other genes that respond to other pathways were not significantly altered. These results demonstrate that KY 19382 promotes chondrocyte proliferation and differentiation via specific activation of the Wnt/β-catenin pathway.

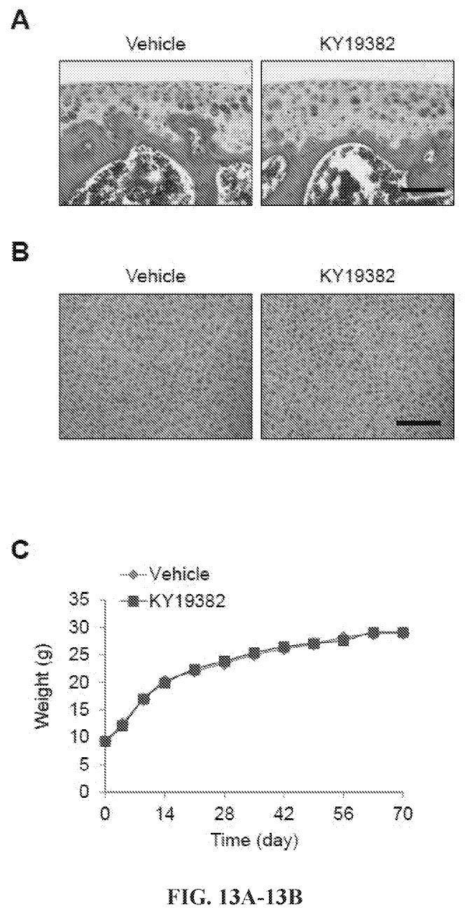

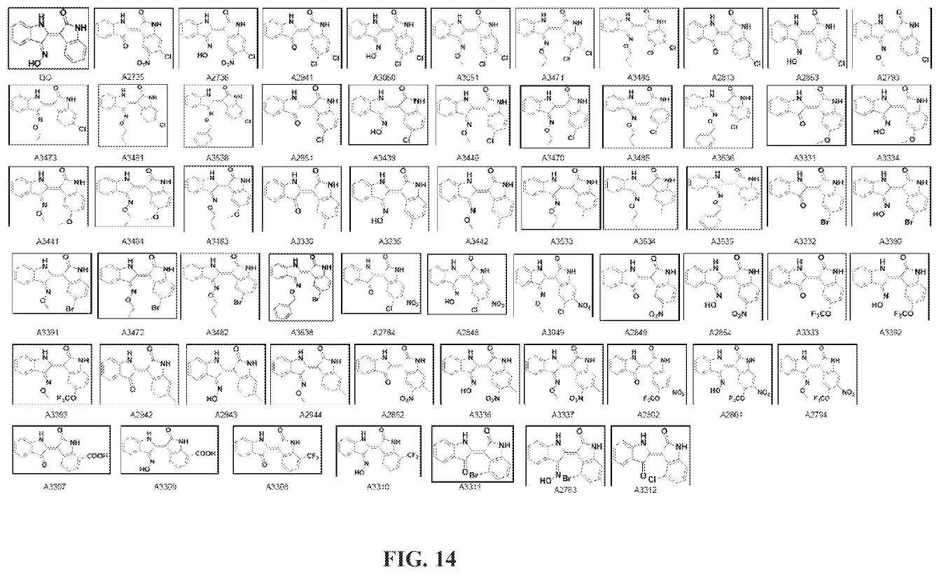

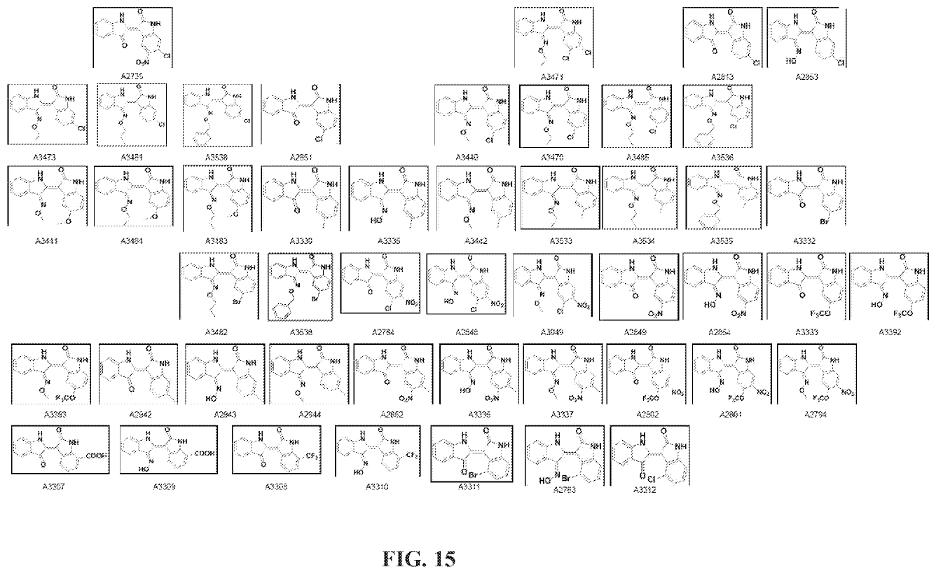

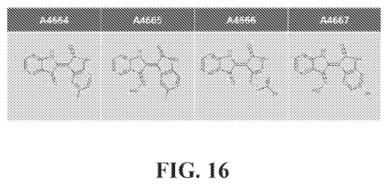

To investigate comprehensive effects from pre- and early puberty to the adulthood period, a long-term administration of KY19382 for 10 weeks in mice from the age of 3 weeks to 13 weeks was performed. Daily treatment of 0.1 mg/kg KY19382 significantly increased the length of tibiae compared to the vehicle-treated group (FIG. 5J). Referring now to FIG. 13, no histological abnormalities were detected in the articular cartilage and the liver tissues of KY19382-treated mice (FIGS. 13A and 13B). During the 10 weeks of treatment, no difference in weight was observed among the groups (FIG. 13C). Taken together, these data reveal that KY19382 induces longitudinal bone growth by promoting growth plate maturation in rapidly growing young mice as well as delaying growth plate senescence in older mice, without noticeable toxicity

The instant disclosure provides that a negative feedback regulator of the Wnt/β-catenin pathway, CXXC5, gradually increased with suppression of β-catenin in growth plate chondrocytes at the later stages of puberty. Upregulation of CXXC5 was in part mediated by estrogen, a sex hormone that can be increased with pubertal progression. Further, the abolishment of estrogen-derived growth plate senescence was observed in Cxxc5^{-/-} mice, and further characterized a role of CXXC5 as a mediator in the estrogen-induced growth plate senescence and subsequent termination of longitudinal bone growth. The function of CXXC5 is exerted to inhibit of Wnt/β-catenin signaling, as shown by both in vitro and in vivo studies that correlates with the inverse relationship of the expression patterns of CXXC5 and β-catenin in the chondrocytes of the growth plate during the process of aging.

CXXC5 can be localized to the cytosol or the nucleus depending on cell type and tissue (Kim et al., 2014; Lee et al., 2015). As disclosed herein, an increase cytosolic CXXC5 during growth plate senescence supports that CXXC5 can exert its function through the interaction with DVL in the cytosol. Unlike CXXC5, CXXC4 (a protein structurally and functionally similar to CXXC5) was not significantly expressed in the growth plate during pubertal progression, indicating that CXXC5 plays a specific role in growth plate senescence.

As cytosolic CXXC5 functions via interaction with PDZ domain of DVL, the CXXC5-DVL interaction was identified and validated herein as a target for the development of drugs that delay growth plate senescence with the use of the PTD-DBMP, a CXXC5-DVL blocking peptide. To further develop small molecules capable of inducing longitudinal bone growth by delaying growth plate senescence, small molecule libraries were screened using an in vitro screening system that monitors the CXXC5-DVL interaction. The indirubin analogs, BIO and I3O, which can act as GSKβ inhibitors, were identified as potential CXXC5-DVL inhibitors.

Development of functionally improved indirubin derivatives, especially KY19382, confirmed that these family compounds can have the dual roles as inhibitors of CXXC5-DVL interaction and/or GSK3β activity. Results disclosed herein showed that KY19382 effectively increased the longitudinal growth of tibiae by delaying growth plate senescence through the accompanying promotion of chondrocyte proliferation and differentiation. While not wishing to be bound by any theory, the effectiveness of KY19382 in enhancing longitudinal bone growth may be due to dual functions via enhancement of growth plate maturation in the rapidly growing young period by inactivation of GSK3β and delay of growth plate senescence in the late pubertal period by interference of CXXC5-DVL interaction.

KY19382 did not exhibit any significant off-target effects as observed by the lack of significant activation of 19 other pathway-specific genes, except the Wnt/β-catenin pathway-target genes. Furthermore, any adverse effects on articular cartilage were not observed following administration of 0.1 mg/kg KY19382 which induced longitudinal bone growth. Targeting cytosolic CXXC5, which functions via the interaction with DVL, can provide additional benefits as this approach will likely reduce any undesirable side effects that can rise from targeting nuclear CXXC5.

TABLE 9
Pharmacokinetic Evaluation of KY19382

| | IV, 1 mg/kg | | IP, 5 mg/kg | |
|---|---|---|---|---|
| PK Parameters | mean | SD | mean | SD |
| tₘₐₓ (hr) | N/A | — | 1.00 | 0.00 |
| Cₘₐₓ (ng/mL) | N/A | — | 463.37 | 29.41 |
| AUCₗₐₛₜ (ng•hr/mL) | 7832.81 | 651.28 | 6555.79 | 572.85 |
| CL (L/hr/kg) | 0.12 | 0.01 | 0.47 | 0.03 |
| Vₛₛ (L/Kg) | 0.33 | 0.07 | N/A | |
| t_{1/2} (hr) | 3.33 | 1.34 | 16.20 | 3.86 |
| F (%) | N/A | — | 16.74 | — |

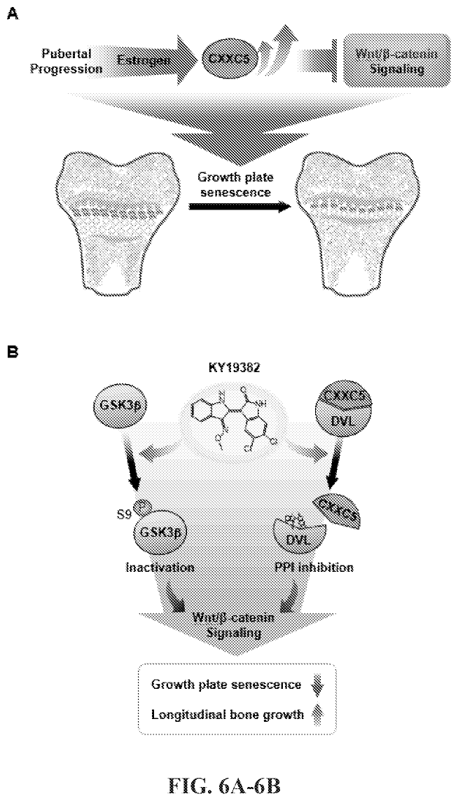

Results disclosed herein provides that estrogen-induced CXXC5 during pubertal progression plays a critical role in promoting growth plate senescence and inhibiting longitudinal bone growth through inactivation of Wnt/β-catenin signaling (FIG. 6A). An effective approach using small molecules that can activate Wnt/β-catenin signaling via a dual mechanism of inhibiting GSK3β and disrupting CXXC5-DVL interaction can be a novel therapeutic strategy for children with growth retardation that involves early growth plate senescence (FIG. 6B).

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety to the extent they are not inconsistent with the explicit teachings of this specification.

We claim:

1. A compound of Formula I, 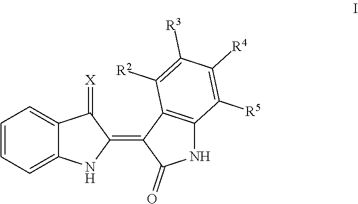
wherein:
X is O or N optionally substituted with R¹;
R¹ is hydrogen, hydroxy, alkyl, alkenyl, or alkoxy optionally substituted with alkyl, alkenyl, haloalkyl, aryl, or benzyl; or R¹ is hydrogen, alkyl, alkenyl, or an alkoxy substituted with butyl, alkenyl, haloalkyl, aryl, or benzyl; and
R², R³, R⁴ and R⁵ are independently hydrogen, nitro, halogen, alkyl, alkenyl, haloalkyl, alkoxy, haloalkoxy, or carboxy.

2. The compound according to claim 1, wherein X is N and R¹ is hydroxy or alkoxy optionally substituted with alkyl, alkenyl, haloalkyl, aryl, or benzyl.

3. The compound according to claim 1 or claim 2, wherein R¹ is alkoxy optionally substituted with alkyl, alkenyl, haloalkyl, aryl, or benzyl.

4. The compound according to any one of claims 1-3, wherein the compound is

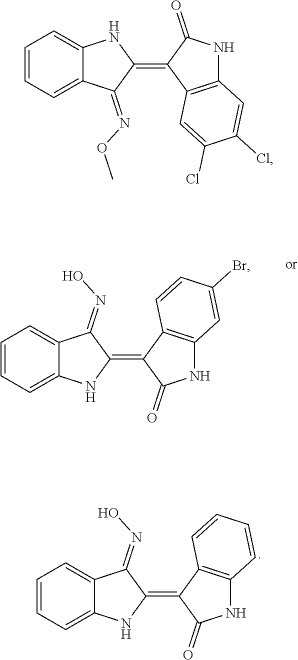 [SMILES: CO/N=C1/C(=C2/C(=O)Nc3cc(Cl)c(Cl)cc32)Nc2ccccc21 ; O=C1Nc2cc(Br)ccc2/C1=C1/Nc2ccccc2/C1=N\O ; "or" ; O=C1Nc2ccccc2/C1=C1/Nc2ccccc2/C1=N\O]

5. A compound of Formula II, 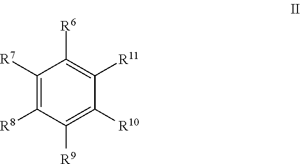
wherein:
R⁶, R⁷, R⁸, R⁹, and R¹⁰ are independently hydrogen, halogen, hydroxy, alkyl, haloalkyl, alkoxy, or 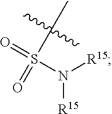
R¹¹ is C₁-C₆ alkyl, C₁-C₆ alkenyl, N, diimide, each substituted with R¹², 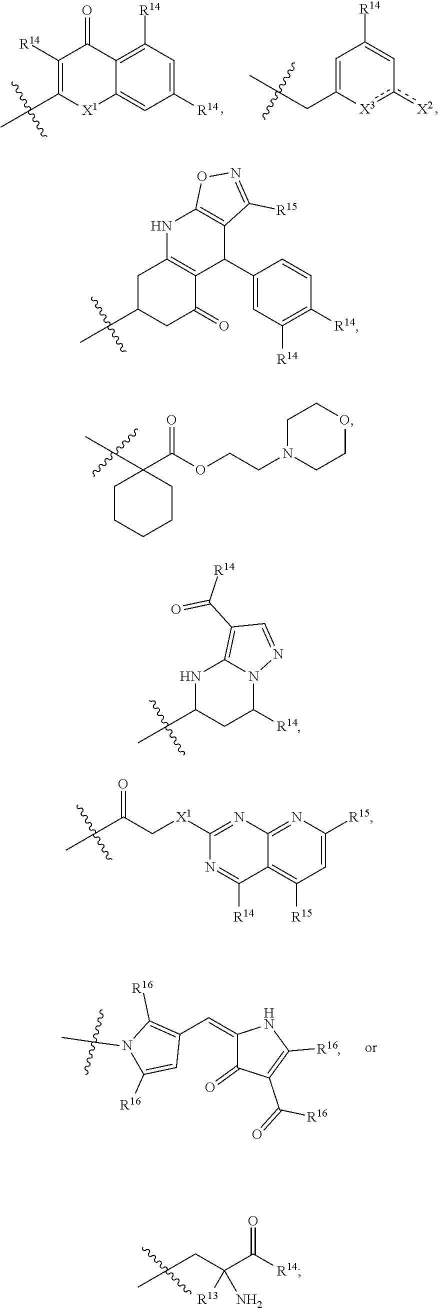
R¹² is 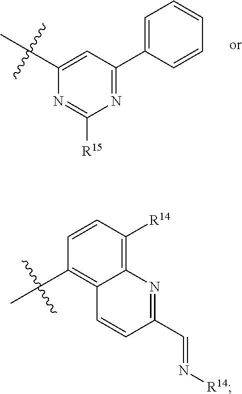
R¹³ is hydrogen or alkyl optionally substituted with hydrogen, halogen, hydroxy, alkoxy, or haloalkyl;
each R¹⁴, each R¹⁵, and each R¹⁶ are independently hydrogen; halogen; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl;
X¹, X² and X³ are independently carbon, nitrogen, oxygen, or sulfur.

6. The compound according to claim 5, wherein the compound is

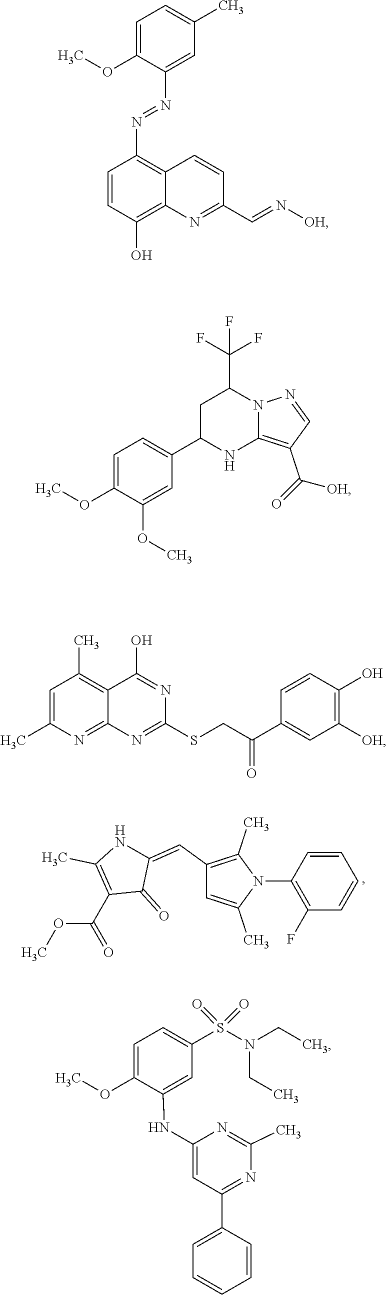 [SMILES: COc1ccc(C)cc1/N=N/c1ccc(O)c2nc(/C=N/O)ccc12 ; COc1ccc(C2CC(C(F)(F)F)n3ncc(C(=O)O)c3N2)cc1OC ; Cc1cc(C)c2c(O)nc(SCC(=O)c3ccc(O)c(O)c3)nc2n1 ; COC(=O)C1=C(C)N/C(=C/c2cc(C)n(-c3ccccc3F)c2C)C1=O ; CCN(CC)S(=O)(=O)c1ccc(OC)c(Nc2cc(-c3ccccc3)nc(C)n2)c1]

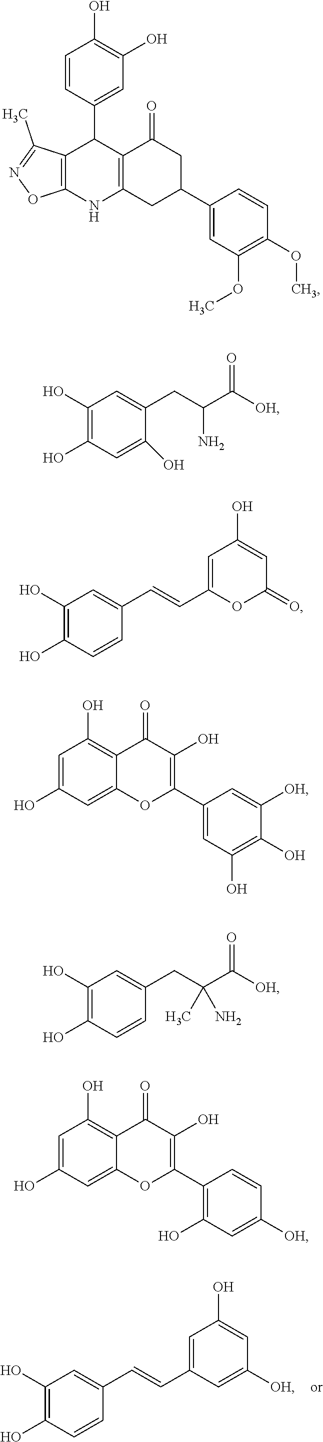

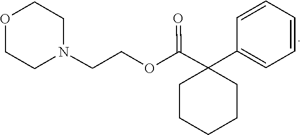 [SMILES: O=C(OCCN1CCOCC1)C1(c2ccccc2)CCCCC1]

7. A compound of Formula III, 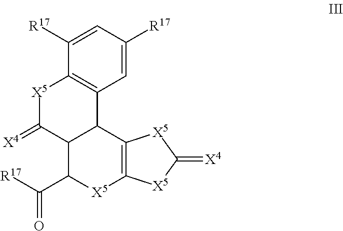
wherein:
each R¹⁷ is independently hydrogen; halogen; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl;
X⁴ and X⁵ are independently nitrogen, oxygen, or sulfur.

8. The compound according to claim 7, wherein each R¹⁷ is independently halogen or hydroxy.

9. The compound according to claim 7 or claim 8, wherein the compound is

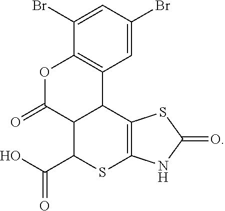 [SMILES: O=C(O)C1Sc2[nH]c(=O)sc2C2c3cc(Br)cc(Br)c3OC(=O)C12]

10. A compound of Formula IV, 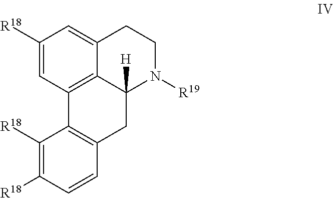
wherein:
each R¹⁸ and R¹⁹ are independently hydrogen; halogen; hydroxy; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl.

11. The compound according to claim 10, wherein the compound is

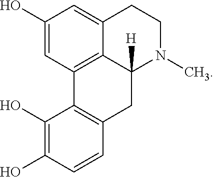

12. A compound of Formula V, 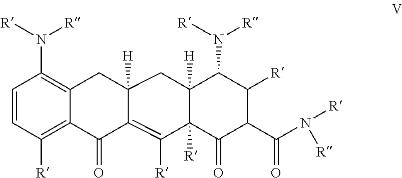
wherein:
each R′ and each R″ are independently hydrogen; halogen; haloalkyl optionally substituted with hydrogen, halogen, hydroxy, or alkoxy; alkyl optionally substituted with hydrogen, halogen, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl.

13. The compound according to claim 12, wherein the compound is

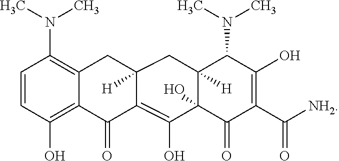

14. A compound of Formula VI, 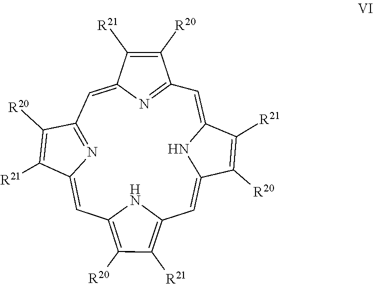
wherein:
each R²⁰ and each R²¹ are independently hydrogen; halogen; haloalkyl optionally substituted with hydrogen, halogen, hydroxy, or alkoxy; alkyl optionally substituted with hydrogen, halogen, hydroxy, alkoxy, haloalkyl, or a carbonyl, wherein the carbonyl is optionally substituted with hydrogen, halogen, alkyl, hydroxy, alkoxy, or haloalkyl; alkoxy optionally substituted with hydrogen, halogen, hydroxy, alkoxy, or haloalkyl; alkenyl optionally substituted with hydrogen, halogen, hydroxy, alkoxy, or haloalkyl; or alkynyl optionally substituted with hydrogen, halogen, hydroxy, alkoxy, or haloalkyl.

15. The compound according to claim 14, wherein the compound is

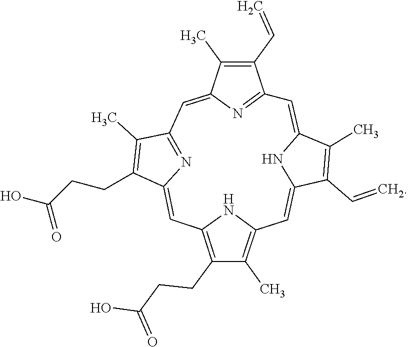 [SMILES: C=CC1=C(C)c2cc3nc(cc4[nH]c(cc5[nH]c(cc1n2)c(C)c5C=C)c(C)c4CCC(=O)O)C(CCC(=O)O)=C3C]

16. The compound according to any one of the claims 1-15, wherein the compound inhibits CXXC5-DVL interface.

17. A pharmaceutical composition comprising at least one compound according to any one of claims 1-16 and/or a pharmaceutically acceptable hydrate, salt, metabolite, or carrier thereof.

18. A method of treating a growth-related disease or a similar condition, comprising:
administering to a subject at least one therapeutically effective dose of at least one agent that reduces and/or inhibits the CXXC5-DVL interaction; or
administering to a subject at least one therapeutically effective dose of at least one agent comprising at least one compound according to claims 1-16 and/or at least one composition according to claim 17.

19. The method according to claim 18, further comprising:
detecting upregulated expression of CXXC5 in the subject.

20. The method according to claim 18 or claim 19, further including the step of:
identifying the subject as at risk for a growth-related disease or a similar condition.

21. The method according to any one of claims 18-20, wherein the subject exhibits abnormal growth plate senescence.

22. The method according to any one of the claims 18-21, wherein the subject is diagnosed with precocious puberty.

23. The method according to any one of claims 18-22, wherein the at least one agent that reduces and/or inhibits the CXXC5-DVL interaction comprises at least one compound according to claims 1-16 and/or at least one composition according to claim 17.

24. The method according to any one of claims 18-23, wherein the subject is a human or an animal.

25. The method according to any one of claims 18-24, wherein the at least one agent is administered orally or intravenously.

26. A method of detecting one or more growth-related disease markers in a subject, comprising:
providing a sample of blood, cells, or tissue from a subject; and
detecting one or more markers in the sample, wherein the one or more markers comprise estrogen and/or CXXC5.

27. The method according to claim 26, wherein the growth-related disease is precocious puberty.

28. The method according to claims 26-27, wherein CXXC5 is overexpressed in the subject.

29. A method of suppressing the activity of CXXC5, comprising:
providing a subject at least one therapeutically effective dose of at least one compound according to any one of claims 1-16, or a pharmaceutically acceptable salt or metabolite thereof.


SEQUENCE LISTING (37 sequences)

SEQ ID NO: 1 - RNA, 25 nt, Artificial Sequence
  Ctnnb1 siRNA-1, sense
auuacaauccgguugugaacguccc

SEQ ID NO: 2 - RNA, 25 nt, Artificial Sequence
  Ctnnb1 siRNA-1, anti-sense
gggacguucacaaccggauuguaau

SEQ ID NO: 3 - RNA, 25 nt, Artificial Sequence
  Ctnnb1 siRNA-2, sense
uaaugaaggcgaacggcauucuggg

SEQ ID NO: 4 - RNA, 25 nt, Artificial Sequence
  Ctnnb1 siRNA-2, anti-sense
cccagaaugccguucgccuucauua

SEQ ID NO: 5 - DNA, 20 bp, Artificial Sequence
  ACTB Forward Primer
agagctacgagctgcctgac

SEQ ID NO: 6 - DNA, 19 bp, Artificial Sequence
  ACTB Reverse Primer
agcactgtgttggcgtaca

SEQ ID NO: 7 - DNA, 23 bp, Artificial Sequence
  COL2A1 Forward Primer
tggaaagcctggtgatgatggtg

SEQ ID NO: 8 - DNA, 22 bp, Artificial Sequence
  COL2A1 Reverse Primer
tgacctttgacaccaggaaggc

SEQ ID NO: 9 - DNA, 22 bp, Artificial Sequence
  MMP13 Forward Primer
gaagacctccagtttgcagagc

SEQ ID NO: 10 - DNA, 22 bp, Artificial Sequence
  MMP13 Reverse Primer
ttcaggattcccgcgagatttg

SEQ ID NO: 11 - DNA, 24 bp, Artificial Sequence
  RUNX2 Forward Primer
caccttgaccataaccgtcttcac

SEQ ID NO: 12 - DNA, 24 bp, Artificial Sequence
  RUNX2 Reverse Primer
catcaagcttctgtctgtgccttc

SEQ ID NO: 13 - DNA, 23 bp, Artificial Sequence
  VEGFA Forward Primer
agggcagaatcatcacgaagtgg

SEQ ID NO: 14 - DNA, 22 bp, Artificial Sequence
  VEGFA Reverse Primer
gtctcgattggatggcagtagc

SEQ ID NO: 15 - DNA, 20 bp, Artificial Sequence
  Actb Forward Primer
ggatgcagaaggagattact

SEQ ID NO: 16 - DNA, 21 bp, Artificial Sequence
  Actb Reverse Primer
ccgatcccacacagagtactt

SEQ ID NO: 17 - DNA, 20 bp, Artificial Sequence
  Alp Forward Primer
gggactggtactcggataac

SEQ ID NO: 18 - DNA, 20 bp, Artificial Sequence
  Alp Reverse Primer
ctgatatgcgatgtccttgc

SEQ ID NO: 19 - DNA, 19 bp, Artificial Sequence
  Col2a1 Forward Primer
gcctgtctgcttcttgtaa

SEQ ID NO: 20 - DNA, 18 bp, Artificial Sequence
  Col2a1 Reverse Primer
tgcggttggaaagtgttt

SEQ ID NO: 21 - DNA, 18 bp, Artificial Sequence
  Col10a1 Forward Primer
tccactcgtccttctcag

SEQ ID NO: 22 - DNA, 20 bp, Artificial Sequence
  Col10a1 Reverse Primer
tttagcctacctccaaatgc

SEQ ID NO: 23 - DNA, 22 bp, Artificial Sequence
  Ctnnb1 Forward Primer
acaagccacaagattacaagaa

SEQ ID NO: 24 - DNA, 21 bp, Artificial Sequence
  Ctnnb1 Reverse Primer
gcaccaatatcaagtccaaga

SEQ ID NO: 25 - DNA, 20 bp, Artificial Sequence
  Gapdh Forward Primer
acccagaagactgtggatgg

SEQ ID NO: 26 - DNA, 20 bp, Artificial Sequence
  Gapdh Reverse Primer
ggatgcagggatgatgttct

SEQ ID NO: 27 - DNA, 20 bp, Artificial Sequence
  Mmp9 Forward Primer
tgaagtctcagaaggtggat

SEQ ID NO: 28 - DNA, 20 bp, Artificial Sequence
  Mmp9 Reverse Primer
atggcagaaataggctttgt

SEQ ID NO: 29 - DNA, 19 bp, Artificial Sequence
  Mmp13 Forward Primer
taagacacagcaagccaga

SEQ ID NO: 30 - DNA, 20 bp, Artificial Sequence
  Mmp13 Reverse Primer
cacatcagtaagcaccaagt

SEQ ID NO: 31 - DNA, 22 bp, Artificial Sequence
  Runx2 Forward Primer
aaggacagagtcagattacaga

SEQ ID NO: 32 - DNA, 18 bp, Artificial Sequence
  Runx2 Reverse Primer
gtggtggagtggatggat

SEQ ID NO: 33 - DNA, 20 bp, Artificial Sequence
  Sox9 Forward Primer
aactggaaacctgtctctct

SEQ ID NO: 34 - DNA, 18 bp, Artificial Sequence
  Sox9 Reverse Primer
acaacacacgcacacatc

SEQ ID NO: 35 - DNA, 26 bp, Artificial Sequence
  Vegfa Forward Primer
ttatttattggtgctactgtttatcc

SEQ ID NO: 36 - DNA, 23 bp, Artificial Sequence
  Vegfa Reverse Primer
tctgtatttctttgttgctgttt

SEQ ID NO: 37 - protein, 26 aa, Artificial Sequence
  phospho-GS2 peptide
YRRAAVPPSPSLSRHSSPHQSEDEEE